(19) United States
(12) Patent Application Publication
(10) Pub. No.: US 2024/0009310 A1
(43) Pub. Date: Jan. 11, 2024

(54) A CHIMERIC ANTIGEN RECEPTOR CONSTRUCT ENCODING A CHECKPOINT INHIBITORY MOLECULE AND AN IMMUNE STIMULATORY CYTOKINE AND CAR-EXPRESSING CELLS RECOGNIZING CD44v6

(71) Applicant: Charité - Universitätsmedizin Berlin, Berlin (DE)

(72) Inventors: Gabriele PECHER, Berlin (DE); Martin RAFTERY, Bad Homburg (DE)

(21) Appl. No.: 18/042,550

(22) PCT Filed: Aug. 24, 2021
(86) PCT No.: PCT/EP2021/073369

§ 371 Date: Feb. 22, 2023

(30) Foreign Application Priority Data
Aug. 24, 2020 (EP) 20192399.2
Nov. 4, 2020 (EP) 20205696.6

(51) Int. Cl. A61K 39/00; C07K 14/725; C07K 14/54; C07K 14/705; C07K 16/28; C12N 5/074; C07K 16/30

(52) U.S. Cl. CPC A61K 39/464428; C07K 14/7051; C07K 14/5443; C07K 14/70521; C07K 16/2884; C12N 5/0696; A61K 39/461; A61K 39/4631; A61K 39/4636; A61K 39/4635; C07K 16/3061; C07K 2319/03; C07K 2319/02; A61K 2239/48

(57) ABSTRACT

A recombinant nucleic acid expression construct including a first nucleic acid sequence region encoding a chimeric antigen receptor (CAR), a second nucleic acid sequence region encoding a checkpoint inhibitory molecule, and a third nucleic acid sequence region encoding an immune stimulatory cytokine. A recombinant nucleic acid expression construct encoding the CAR specifically recognizes CD44v6, and includes a PD1 checkpoint inhibitory molecule, and an immune stimulating cytokine. Further aspects relate to genetically modified cells, including a recombinant nucleic acid expression construct encoding the CAR, wherein the cells are preferably immune cells, more preferably NK cells or cytotoxic T lymphocytes or T helper cells. Medical use of the cells may be in the treatment of a medical disorder associated with the presence of pathogenic cells expressing CD44v6, preferably cancer cells, more preferably cancer stem cells of solid or liquid malignancies.

The invention relates to a recombinant nucleic acid expression construct comprising a first nucleic acid sequence region encoding a chimeric antigen receptor (CAR), a second nucleic acid sequence region encoding a checkpoint inhibitory molecule, and a third nucleic acid sequence region encoding an immune stimulatory cytokine. The invention further relates to a recombinant nucleic acid expression construct encoding the CAR of the invention that specifically recognizes CD44v6, and comprises a PD1 checkpoint inhibitory molecule, and an immune stimulating cytokine. In further aspects, the invention relates to genetically modified cells, comprising a recombinant nucleic acid expression construct encoding said CAR, wherein the cells are preferably immune cells, more preferably NK cells or cytotoxic T lymphocytes or T helper cells. The invention further relates to corresponding medical uses of said cells in the treatment of a medical disorder associated with the presence of pathogenic cells expressing CD44v6, preferably cancer cells, more preferably cancer stem cells of solid or liquid malignancies.

### BACKGROUND OF THE INVENTION

Targeting the vulnerabilities of cancer cells—that is the goal of personalized cancer therapies, also known as “personal medicine” or “targeted therapy”. Adoptive T cells immunotherapy is considered a promising anti-tumor treatment. Genetically modified T cells expressing chimeric antigen receptors (CARs) eliminate tumor cells by binding to tumor antigens through antigen-antibody recognition. These chimeric antigen receptor T (CAR-T) cells directly recognize tumor cells without the process of antigen presentation and without being restricted by the major histocompatibility complex (MHC). With CAR-T cell therapeutics, a promising gene therapy concept has been introduced into hematology and oncology.

CAR-T therapy revealed an encouraging anti-tumor effect and a high rate of complete remissions in hematological CD19+ malignancies. Researchers have performed CAR-T therapy applications in solid tumors by targeting multiple tumor-associated antigens, such as human epidermal growth factor receptor 2 (HER2), carboxylic acid anhydrase IX (CAIX), carcinoembryonic antigen (CEA), disialoganglioside (GD2), and interleukin (IL)-13-receptor alpha 2 (IL-13Rα2).

Advantages of the CAR technology are based on the fact that the patient autologous T cells or the donor allogenic T cells are genetically engineered ex vivo in the laboratory with a recombinant surface molecule, which on the one hand has specificity for tumor cells and on the other hand mediates activation of the T cells when the tumor cells is recognized. The prototype of this surface molecule has in the extracellular part an antibody, preferably a scFv single chain antibody for the recognition of an antigen on tumor cells, and in the intracellular part a signal chain for T cell activation, preferably the CD3ζ chain of the T cell receptor (TCR). A transmembrane domain anchors the molecule in the cell membrane. Since the surface molecule is composed of an antibody (fragment) on the one hand and a T cell signalling unit on the other, it is referred to as a “chimeric antigen receptor” (CAR). The CARs are therefore recombinant surface receptors which, in contrast to the natural T cell receptors (TCR), recognize their target antigens by means of an antibody and independently of the HLA (human leukocyte antigen) complex. The genetic information for the respective CAR has so far mostly been introduced into the patient's or donor's T cells by means of retro- or lentiviral vectors by transduction ex vivo, which then express the CAR on the surface. The CAR-T cells are reinfused into the patient and recognize specific antigens that are formed on the surface by cancer cells. A “first generation” CAR has a signalling chain for primary T cell activation, usually the CD3 signalling chain; a “second generation” CAR uses the CD3ζ chain together with a costimulatory unit, preferably from the CD28 family, to provide a sustained T cell activation.

Although some efficacy was observed in certain studies with these CAR generations, including complete regression of a single glioblastoma case treated with multiple infusions of IL-13Rα2 CAR-T cells, the overall outcome of CAR-T cell therapy, in particular in solid tumors, was not sufficient in a large number of studies. Another important issue of CAR-T therapy are adverse events of a cytokine release syndrome and on-target/off-tumor effects. CAR-T therapy can also cause neurologic side effects such as speech problems, tremors, delirium, and seizures.

Therefore, a CAR re-design with the goal of creating a safer and more effective therapy is needed to improve safety and efficacy of the CAR-T cell therapy.

There are some aspects critical for an effective CAR-T cell strategy in the treatment of solid tumors. Among the antigens of solid tumors, that CAR-T cell therapy targets, are CD44v6, CEA, EGFR, EGFRvIII, GD2, HER2, IL13Rα2, PSCA, Tn-MUC1, and PSMA. While all these antigens are either overexpressed and/or amplified in tumors compared to normal tissue, their protein expression is not clearly restricted to tumor cells, with the exception of EGFRvIII, a common oncogenic rearrangement in glioblastoma characterized by deletion of exons 2-7 of EGFR. Therefore, in contrast to CD19, a B-cell restricted antigen expressed in many B-cell malignancies, antigen targets of solid tumors give rise to significant concerns about toxicity, which may limit their usefulness in CAR-T cell therapy. Thus, tumor specificity of the antigen selected is one critical aspect in CAR design for an effective and safe CAR-T cell strategy.

Another aspect well established is tumor resistance to individual therapeutics, since the majority of tumors are heterogeneous. Prolonged targeting of a single drug-sensitive pathway may ultimately lead to drug-resistant tumor recurrence and escape variants. Acquired or intrinsic resistance patterns have been observed after CAR-T cell therapy. CD19 CAR-T cell therapy has shown sustained clinical remissions in 70-90% of patients with B-cell malignancies, including acute lymphoblastic leukemia (ALL), but recent follow-up data from clinical trials show a common mechanism of resistance, including loss and/or downregulation of CD19 antigen in up to 70% of patients who relapse after treatment. Early clinical findings using CAR-T cells in solid tumors have observed similar resistance mechanisms of antigen leakage. There is strong evidence that a rational design combining tumor specific antigen targeting with other anti-tumor strategies will be necessary for effective disease control.

An immunosuppressive tumor microenvironment is another challenge for an effective immunotherapy using CAR. Unlike most hematological malignancies, there are no local immunosuppressive pathways that impede anti-tumor immunity and limit adoptive T cell therapies. Solid tumors can be strongly infiltrated by multiple cell types that support tumor growth, vascularization, and metastasis and may control therapeutic responses. In addition to the tumor cells themselves, the immune cells, such as regulatory T cells and myeloid suppressor cells, induce local cytokine, chemokine, and growth factor production in solid tumors, including IL-4, IL-10, VEGF and TGFβ. Similarly, immune checkpoint pathways, including PD-1 and CTLA-4, can be highly active in tumors to attenuate anti-tumor immunity. There is strong evidence that the microenvironment of the tumor controls the response and resistance to immune therapies and may limit the effectiveness of CAR-T cell therapy.

CD44 is a transmembrane glycoprotein and is mainly expressed on epithelial and mesenchymal cells. One CD44 tissue-specific isoform is CD44v6. CD44v6 is involved in cell growth, apoptosis, migration and angiogenesis and interacts with hyaluronic acid and growth factors (VEGF, EGF). It is a known tumor marker, which is highly expressed in solid tumors and induces cancer. Since CD44v6 is involved in Wnt activation and is mainly expressed on tumor stem cells, it plays a crucial role in metastasis and is associated with a fatal prognosis for patients with solid tumors. Currently, no curative therapy exists for CD44v6 high-positive tumor stem cells.

Alternative treatment options for solid cancers in general currently include surgery to remove the tumors, chemotherapy, interleukins, checkpoint inhibitors, specific antibodies against surface proteins. For CD44 positive tumor diseases, the monoclonal antibody against CD44 and CD44v6 is tested in clinical trials. A CAR-T technology system against solid tumors does not exist so far.

Many laboratories have long tried unsuccessfully to functionalize such a CAR combination and to solve the above-mentioned technical difficulties. To the knowledge of the inventors, no equivalent and functional anti-CD44v6 CAR constructs combined with an effective immune stimulatory cytokine and checkpoint inhibitory molecule have been previously described, and no anti-CD44v6 antibody studies relevant to the medical indication of the present invention are currently available.

A major concern with CAR-T therapy is the danger of a “cytokine storm” associated with intense antitumor responses mediated by large numbers of activated T cells (Sadelain et al., Cancer Discov 3:388-98, 2013). Side effects can include high fever, hypotension and/or organ failure, potentially resulting in death. The cytokines produced by CAR-NK cells differ from CAR-T cells, reducing the risk of an adverse cytokine-mediated reaction.

Therefore, improving CAR immunotherapy requires overcoming complex technical and biological problems. The present invention addresses the three most challenging areas requiring attention in CAR vector design and the development of next-generation CARs for solid tumors, namely (1) the targeting of tumor specific antigens, (2) the heterogeneity of tumor antigens and escape, and (3) the immunosuppressive tumor microenvironment. The invention described below relates to the solution of this problem.

### SUMMARY OF THE INVENTION

In light of the prior art, the technical problem underlying the present invention is to provide a novel strategy for immunotherapy of cancer. In particular, a problem underlying the invention is the provision of suitable means for local stimulation of a targeted immune response directed against a tumor tissue in a subject whilst avoiding tumor escape from immunotherapy. A further problem underlying the invention is the provision of means for a check point inhibitor and stimulation of the immune response that provides an effective local effect at the targeted tumor tissue with reduced levels of unwanted side effects.

This problem is solved by the features of the independent claims. Preferred embodiments of the present invention are provided by the dependent claims.

The invention relates to a recombinant nucleic acid expression construct, comprising:
(a.) a first nucleic acid sequence region encoding a chimeric antigen receptor (CAR),
(b.) a second nucleic acid sequence region encoding a checkpoint inhibitory molecule, and
(c.) a third nucleic acid sequence region encoding an immune stimulatory cytokine.

The invention therefore relates to a recombinant nucleic acid expression construct and corresponding immune cells expressing said construct, preferably a CAR-T cell product or CAR-NK cell product that confers human T cells or NK cells with a high cytotoxic activity against defined, solid or liquid tumors, while sparing non-pathogenic cells within the tissue surrounded by the tumor, such as breast, pancreatic, lung, colon or liver cells as well as hematological cells.

In preferred embodiments all T cells, B cells, NK cells are likewise spared; as the CAR-T cell or CAR-NK cell products of the present invention shows no or negligible activity against these cells.

In a preferred embodiment, the immune cells are a T cell. In other embodiments, the immune cells are T cells comprising an artificial T cell receptor, such as a chimeric antigen receptor (CAR-Ts), wherein said T cell receptor binds specifically to a tumor antigen (Lee, D W et al., Clin Cancer Res; 2012; 18(10); 2780-90). In a preferred embodiment, the immune cells are NK cells. In other embodiments, the immune cells are NK cells comprising an artificial NK cell receptor, such as a chimeric antigen receptor (CAR-NKs), wherein said NK cell receptor binds specifically to a tumor antigen.

The present invention enables the stimulation of cells involved in an anti-tumor immune response and thereby the local activation, support and/or strengthening of an anti-tumor immune response. The present invention enables an effective and therapeutically relevant dose of one or more immune stimulatory cytokines to be administered via expression from a recombinant nucleic acid expression construct in transplanted CAR-T cells or CAR-NK cells while avoiding the significant side effects that are inherent in systemic administration of cytokines without an appropriate targeting agent. The invention therefore relates to the utilization of CAR-T cells or CAR-NK cells as a targeting agent and/or vehicle for the local delivery of immune modulatory, preferably immune stimulating signals in regions of inflammation, preferably in and in proximity to tumor tissue.

A crucial limitation in the successful development and medical use of immunotherapies is the ability of tumors to escape and/or suppress the natural immune response against the tumor cells, by establishing an immunosuppressive tumor microenvironment. This phenomenon is known as tumor-mediated immunosuppression and is mediated to a large extent by the secretion of anti-inflammatory cytokines by immune cells and checkpoint proteins present in the tumor that display a regulatory phenotype (for example, regulatory T cells and monocyte-derived suppressor cells). The invention therefore provides means to modify the tumor microenvironment, making it pro-inflammatory, promoting the activation of immune cells present in the tumor and recruitment and activation of external immune cells and thereby facilitating the broad activation of the immune system against the tumor and/or enhance the efficacy of anti-tumor immunotherapeutic treatments.

In one embodiment the recombinant nucleic acid expression construct as described herein can be administered into human cells in order to modify and render the tumor microenvironment favorable and conducive to immunotherapies.

The present invention makes use of CAR-T cells or CAR-NK cells as a cellular vehicle for the delivery of immunomodulatory effectors for simulating an immune response, thereby utilizing the unique tumor antigen targeting effect homing CAR-T cells or CAR-NK cells to the regions of the tumors, and thereby exert local therapeutic effects based on stimulation of an appropriate immune response and inhibiting checkpoint proteins, wherein the immune response relates preferably to the natural immune response of a host (subject), and thereby enhance the efficacy and therapeutic effect of immunotherapeutics, such as chimeric antibodies, adoptive immunotherapies, anti-tumor vaccines and/or checkpoint inhibitors. The invention further provides means to optionally switch off the genetically modified cells product expressing the CAR, as described here, i.e. for safe clinical use of the said cell. This safety feature can be realized, in some embodiments, by an inducible suicide gene.

Surprisingly, the CAR-T cell modified with the recombinant nucleic acid expression construct comprising the nucleic acid sequence region encoding the CAR, one or more immune stimulating cytokine(s), and/or a checkpoint inhibitory molecule as described herein show unexpectedly good expression and secretion of said cytokines and checkpoint inhibitory molecules, both in vitro and in vivo. A skilled person would not expect that these particular cytokines and checkpoint inhibitory molecules could be expressed in sufficient quantities and exported from the cells in sufficient quantities to induce or enhance the desired local immune response, based on either the innate response and/or immunotherapy.

The invention also encompasses the expression of a combination of immune activating cytokine and/or checkpoint inhibitory molecules in tumors via CAR-T cells or CAR-NK cells described herein, with the aim to attract immune effector and helper cells, induce immune activation, promote the maturation of memory immune cells and/or suppress the emergence and persistence of suppressive and/or regulatory immune cells.

In one embodiment, a combination of the CAR targeting a tumor antigen, the immune stimulating cytokine(s), and/or the checkpoint inhibitory molecules is used, in order to promote the activation of different arms of the immune response, including the innate and adaptive immune response, effector, helper, and/or antigen presenting cells.

On the other hand, cytokines such as IL-2, IL-7, IL-15 and IL-21 specifically activate cytotoxic lymphocytes such as T cells and NK cells that mount a specific response against tumor cells. Likewise, IL-15 will activate cytotoxic lymphocytes, but also monocytes and helper cells.

In particular, a problem underlying the invention is the provision of suitable means for local stimulation of a targeted immune response directed against a tumor tissue in a subject with less tumor escape from immunotherapy. A further problem underlying the invention is the provision of means of a check point inhibitor and stimulation of the immune response that provides an effective local effect at the targeted tumor tissue with reduced levels of unwanted side effects.

A combination of the tumor specific CAR, the immune stimulatory cytokine, and the checkpoint inhibitory molecules therefore yields synergistic effects, as or beyond what is seen in the natural immune response, immunotolerant tumor microenvironment, and/or low rate of tumor escape. The present invention increases therapeutic efficacy and tumor regression rates.

It was a surprising result that the natural immune response could, in effect, be mirrored, or analogously applied, in an enhanced manner using a combined CAR-T cells or CAR-NK cells approach. The invention is therefore based on the surprising finding that by providing a combination of transgenes encoding the tumor specific CAR, the immune stimulating cytokine(s), and the checkpoint inhibitory molecule in CAR-T cells or CAR-NK cells, a locally more effective and safe anti-tumor response can be obtained. The combination leads to unique local expression and secretion of the immune-stimulating factors that leads to a local anti-tumor response, comprising multiple arms of the immune response, without inducing systemic toxicity as is often observed when systemically applying cytokines in tumor patients.

Furthermore, the unique properties of this particular CAR-T cell combination as shown in the Examples, which home to and engraft into tumor tissue, leads to maintained expression of the therapeutic tumor specific CAR, immune stimulator cytokine factors, and checkpoint inhibitory molecules in order to maintain the immune response for therapeutic effect.

In one embodiment, the recombinant nucleic acid expression construct of the invention optionally comprises an additional sequence region, otherwise termed a fourth sequence region, encoding a chemokine receptor, such as the chemokine receptor CCR4. Preferably the chemokine receptor enables cell migration to a tumor cell.

Chemokine receptors are cytokine receptors expressed and presented on the surface of cells that interact with a type of cytokine called a chemokine. Chemokines are a family of small cytokines, or signaling proteins secreted by cells and they typically induce directed chemotaxis of responsive cells towards or away from chemokine producing cells; they are often referred to as chemotactic cytokines. The expression of a chemokine receptor is therefore associated with the additional advantage of enhancing cell migration or motility of a cells expressing the CAR of the invention to a target cell.

CCR4 (also termed C—C chemokine receptor type 4 or CD194) belongs to the G protein-coupled receptor family and is a receptor for the CC chemokines CCL2 (MCP-1), CCL4 (MIP-1), CCL5 (RANTES), CCL17 (TARC) and CCL22 (Macrophage-derived chemokine). For example, CCL2 recruits monocytes, T cells, and dendritic cells to sites of inflammation, CCL4 is a chemoattractant for natural killer cells, monocytes and a variety of other immune cells, and CCL5 is chemotactic for T cells, eosinophils, and basophils, also recruiting leukocytes to inflammatory sites. The expression of CCR4 therefore enables enhanced recruitment and/or migration of genetically modified cells expressing the CAR of the invention to tumor tissue.

Other chemokine receptors (such as one or more of CCR1-11) may be considered by a skilled person and selected depending on the tumor type and antigen specificity of the CAR, in order to enhance specific migratory properties of the modified cells.

In one embodiment, the present invention provides CAR expressing T-cells or NK cells expressing more than one immune stimulating and/or immune suppression defeating gene and/or an inducible suicide gene allowing said cells to be destroyed. A suicide gene, as non-limiting embodiments, is one that codes for the thymidine kinase of an alpha herpesvirus (HHV1-3), the bacterial gene cytosine deaminase, which can convert 5-fluorocytosine into the highly toxic compound 5-fluorouracil, and inducible caspase-9 or caspase-8. Inducible caspase-9 can be activated by a specific chemical inducer of dimerization (CID). Suicide genes may also be polypeptides that are expressed on the cell surface and can make the cells sensitive to therapeutic monoclonal antibodies. The suicide gene expression may be inducible, for example by doxycycline adapted to human cells.

In one embodiment, the first nucleic acid sequence region encoding the CAR comprises:
(d.) a nucleic acid sequence encoding an extracellular antigen-binding domain, said antigen-binding domain preferably comprising an antibody or antibody fragment,
(e.) a nucleic acid sequence encoding a transmembrane domain, and
(f.) a nucleic acid sequence encoding an intracellular co-stimulatory domain,

In any of the first nucleic acid sequence region encoding the CAR comprises a nucleic acid sequence encoding an extracellular antigen-binding domain, wherein the antigen binding domain binds to a cancer associated antigen selected from the group consisting of: Folate receptor alpha (FRa), ERBB2 (Her2/neu), EphA2, IL-13Rα2, epidermal growth factor receptor (EGFR), Mesothelin, TSHR, CD19, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, interleukin-11 receptor a (IL-11 Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-I receptor, CAIX, LMP2, gp100, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p⁵³ mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1 B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In a preferred embodiment, the antigen binding domain of the CAR construct recognizes (preferably specifically binds to) CD44v6. Corresponding immune cells expressing said construct, preferably a CAR-T cells or CAR-NK cell product, exhibit a high cytotoxic activity against CD44v6 positive pathogenic cells.

The invention also relates to a cell product, such as an advanced therapy medicinal product, designed and manufactured for medical use, comprising next generation anti-CD44v6 CAR transduced immune cells that can be used in the treatment of cancer.

As shown in the Examples, the CAR target CD44v6 is strongly expressed on MM, Lymphomas, and Leukemias but absent or weakly expressed in normal (untransformed or non-cancerous) cells.

Additional embodiments of the first nucleic acid sequence region encoding the CAR are provided below: In any of the nucleic acids encoding the antigen binding domain of the CAR described herein, the antigen binding domain of the CAR is connected to the transmembrane domain by a hinge region. In any of the nucleic acids encoding the transmembrane domain of the described herein, the transmembrane domain of comprises a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137 and CD154. In any of the nucleic acids encoding the intracellular signaling domain of the CAR described herein, the intracellular signaling domain comprises a costimulatory signaling domain comprising a functional signaling domain obtained from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a.

In preferred embodiments of the immunotherapy approach of the present invention, patient-derived T cells or NK cells are transduced, preferably retrovirally, to express an artificial immune receptor as described herein, composed of an extracellular antibody-derived antigen recognition part, fused to a transmembrane section, and followed by intracellular signaling domains. The construct described herein therefore confers transduced T cells or NK cells with anti-tumor cytolytic capacity.

For the first time, the anti-CD44v6 CAR-T cells will enable targeting of the tumor cells in the tumor microenvironment as shown in the examples described herein.

The tumor antigen specific CAR-T cell, such anti-CD44v6 CAR-T cells, or CAR-NK cells as described herein is in preferred embodiments applicable to the treatment of solid or liquid tumor patients who are not eligible for other therapies. More specifically, embodiments of the invention relate to the treatment of the following patient collectives:
i) patients with multidrug resistances,
ii) patients not eligible for allogeneic stem cell transplantation,
iii) patients with co-morbidities that preclude further chemotherapies,
iv) patients suffering from solid and/or liquid cancers,
v) aged patients who do not tolerate chemotherapies,
vi) the CAR is applicable for salvage therapies even after progressive disease and multiple lines of other standard of care therapies have failed,
vii) it is applicable even at low antigen density on target tumor cells, where antibodies can fail, and/or
viii) it is applicable as a monotherapy which is not the case for antibodies
ix) it is applicable in combination with one or more anti-cancer treatments, anti-cancer medicaments or transplantation.

The anti-CD44v6 CAR described herein confers high avidity to T cells or NK cells, necessary for anti-tumor efficacy. The present invention has an unprecedented low off-target reactivity on other tissues.

As demonstrated in the examples below, in an in vitro co-culture system, anti-CD44v6 CAR-T cells become activated upon exposure to CD44v6-expressing human tumor cell lines. These T cells then develop an effector phenotype with high level of a cytotoxic activity.

Additionally, a cytotoxicity assay against selected target cell lines, CD44v6-negative cells, shows that selective cellular cytotoxicity is obtained only in cell lines positive for CD44v6.

Surprisingly, the combined expression of anti-CD44v6 CAR, the immune stimulatory cytokine IL-15, and the checkpoint inhibitor against PD-1 in Jurkat cells shows a synergistic effect (see examples). A skilled person would expect an additive effect of the combination described herein and not a synergistic effect as shown in the examples. This synergistic effect is unexpected and represent a special technical feature of the invention.

As such, the CAR of the present invention represents a surprising and beneficial approach towards the treatment of the medical conditions described herein. The employment of anti-CD44v6 CARs has not been previously attempted or described as a promising approach towards treating solid or liquid tumors, more preferably positive for CD44v6. The minimal (if not non-existent) unwanted side effects, due to the selectivity of the marker, also represent a beneficial and surprising aspect of the present invention. In particular in patients, in which resistance to other solid or liquid tumor treatments have arisen, the present invention represents a very promising approach towards eradication of malignancies.

Cancer according to the present invention refers to all types of cancer or neoplasm or malignant tumors found in humans. In some embodiments it is advantageous, that the CAR construct in the present invention can be used in therapy of solid tumors and/or liquid tumors, i.e. leukaemia or lymphoma. State of the art CAR constructs can be used to treat either solid or liquid tumors. In some embodiments, the genetically modified cells is used as a medicament, wherein the disorder to be treated with said immune cell are selected from a group of cancers as described herein, preferably consisting of breast cancer, pancreatic tumor, colon carcinoma, and acute myeloid leukemia.

In one embodiment, the extracellular antigen-binding domain of the CAR encoded by the first nucleic acid sequence region specifically recognizes CD44v6.

Examples of CD44v6 binding domains suitable for use in a CAR are further described herein. In one embodiment, the CD44v6 binding domain is a provided in SEQUENCE Table.

Examples of CD44v6-expressing cells are known to a skilled person, and can be identified by further screening of cancers or other pathogenic cells. Cell lines expressing CD44v6 are preferably fHDF/TERT166, U-87 MG, HMC-1, HBEC3-KT, HaCaT, WM-115, hTERT-HME1, and/cells isolated from the proximal digestive tract, the lung, and/or skin.

In one embodiment, at least the first nucleic acid sequence region encoding the CAR is constitutively expressed by a promoter or promoter/enhancer combination, preferably selected from the group consisting of a Spleen Focus Forming Virus (SFFV) promotor, EF1alpha promoter (for example the EF1alphaS promoter), PGK promoter, CMV promoter, SV40 promoters, GAG promoter and UBC promoter, more preferably a Spleen Focus Forming Virus (SFFV) promotor.

In one embodiment, the recombinant nucleic acid expression construct is or may be termed a nucleic acid construct. In some embodiments, said constructs may be provided with or without a promoter. A skilled person is capable of identifying suitable promoters and/or generating constructs with suitable promoters, depending on the intended application.

In a preferred embodiment, the promotor region comprises a constitutive promotor region, e.g., an elongation factor 1 alpha (EF1a) promotor region as described herein. The CAR is operatively linked to promotor region comprising a constitutive promotor region. Due to the beneficial tumor antigen specific properties of CAR-T cells to tumors within the body of a subject, post systemic administration or after local administration, the use of a constitutive promoter for expression of the one or more immune response-stimulating cytokine(s) is preferred.

In one embodiment, at least the first nucleic acid sequence region encoding the CAR and the second nucleic acid sequence region encoding the checkpoint inhibitory molecule, are configured to encode a polycistronic mRNA comprising coding regions for the polypeptide sequences of the CAR and the checkpoint inhibitory molecule, and wherein an amino acid sequence comprising a polypeptide cleavage site is disposed between the CAR polypeptide and the checkpoint inhibitory molecule polypeptide.

In one embodiment, the polypeptide cleavage site is selected from the group consisting of P2A, T2A, E2A and F2A.

In one embodiment, at least the first nucleic acid sequence region encoding the CAR and the second nucleic acid sequence region encoding the checkpoint inhibitory molecule, are configured to encode a polycistronic mRNA comprising coding regions for the polypeptide sequences of the CAR and the checkpoint inhibitory molecule, and wherein an amino acid sequence comprising the polypeptide cleavage site P2A is disposed between the CAR polypeptide and the checkpoint inhibitory molecule polypeptide.

In a preferred embodiment, the checkpoint inhibitory molecule encoded by the second nucleic acid sequence region is a dominant negative polypeptide and/or an antibody inhibiting and/or blocking an immune checkpoint protein, wherein the checkpoint inhibitory molecule is preferably a dominant negative truncated PD1 polypeptide or a PD1-antibody.

Normally, cells that are potentially cancerous are destroyed by the immune system. All cancer cells undergo changes that differentiate them from their neighbors, the most obvious change being the ability to multiply without inhibition. Cancer cells utilize mechanisms that avoid regular immune system control. Checkpoint proteins have been shown to function by communicating to the immune system that a potentially cancerous cell is not to be destroyed. There may be other molecules signaling that the cell is cancerous, but if there are enough checkpoint proteins on the cell surface, the immune system may overlook cancerous signals.

A ligand-receptor interaction that has been investigated as a target for cancer treatment is the interaction between the transmembrane programmed cell death 1 protein (PD-1; also known as CD279) and its ligand, PD-1 ligand 1 (PD-L1). In normal physiology PD-L1 on the surface of a cell binds to PD1 on the surface of an immune cell, which inhibits the activity of the immune cell. It appears that up-regulation of PD-L1 on the cancer cell surface may allow them to evade the host immune system by inhibiting T cells that might otherwise attack the tumor cell. Antibodies that bind to either PD-1 or PD-L1 and therefore block the interaction may allow the T cells to attack the tumor.

Checkpoint inhibitors (also known as immune checkpoint modulators, or CPMs) are designed to lessen the effectiveness of checkpoint proteins. They may have a variety of mechanisms of action, but if effective, they enable the immune system to recognize other molecules on the surface of the cancer cells.

In a preferred embodiment, the medical use of the genetically modified CAR-T cells or CAR-NK cells as described herein is characterized by the combined transgenic expression of a checkpoint inhibitor, preferably a PD-L1 and/or PD-1 inhibitor, with said CAR and immune-stimulatory cytokine.

In a preferred embodiment, the checkpoint inhibitory molecule is a dominant negative truncated form of PD-1 (dnPD1opt). DnPD1opt binds to the native form of PD-1 and therefore blocks the PD-1 protein.

In one embodiment, the third nucleic acid sequence region encoding an immune stimulatory cytokine (also referred to as an immune-stimulating cytokine, or as an immune-response stimulating cytokine) comprises a nucleic acid sequence encoding one or more immune response-stimulating cytokines operably linked to one or more promoters, wherein at least one of said cytokines is selected from the group consisting of IL-15, IL-15RA, IL-2, IL-7, IL-12, IL-21, IFN gamma and IFN beta. Cytokines that stimulate the immune response are known to a skilled person and can be assessed and/or determined by established routine assays.

The present invention encompasses in some embodiments the combination of cytokine, checkpoint inhibitory molecule and tumor specific CAR transgenes in the cells as described herein, in particular any given specific combination of said cytokine disclosed herein, preferably any given specific combination of one or more of IL-15, IL-15RA, IL-2, IL-7, IL-12, IL-21, IFN gamma, IFN beta, CD28. The CAR-T cells or CAR-NK cells described in this invention, defined by at least one immune-stimulating cytokine and the method of tumor treatment comprising an ex vivo production of an allogenic CAR-T cells or CAR-NK cells construct as described herein and the transfer of the allogenic CAR-T cells or CAR-NK cell product into a patient, preferably in combination with other anti-tumor immunotherapies, are bound by the surprising and beneficial concept of local immune system stimulation in an anti-tumor immune response, either by the innate immune system or by combined immunotherapies. It was unexpected that CAR-T cells encoding transgenic immune-stimulating cytokines as shown in the examples may be used as an effective anti-tumor adjuvant in stimulating an anti-tumor response. However, the use of CAR-T cells or CAR-NK cells additionally encoding transgenic immune-stimulatory cytokines and checkpoint inhibitory molecules, to boost the local anti-tumor immune response, represent special technical features of the invention.

In a preferred embodiment the genetically modified mesenchymal stem cells as described herein is characterized in that the immune response-stimulating cytokine is IL-15.

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 induces cell proliferation of natural killer cells; cells of the innate immune system whose principal role is to kill virally infected or cancerous cells. IL-15 has been shown to enhance the anti-tumor immunity of CD8+ T cells in pre-clinical models (Klebanoff C A, et al. Proc. Natl. Acad. Sci. U.S.A. 101 (7): 1969-74).

The present invention enables a surprising and advantageous anti-tumor effect via the expression of an immune stimulatory cytokine in CAR-T cells as described herein. The expression of a stimulatory cytokine as described herein by the CAR-T cell supports an anti-tumor immune response and leads to reduction in tumor size and/or growth, and shows a distinct reduction in and/or avoidance of the side effects produced by systemic administration of such cytokines known in the art. Side effects such as nausea and vomiting, sores in the mouth or on the lips, diarrhea, drowsiness, allergic reactions, fever or chills, hives, itching, headache, coughing, shortness of breath, or swelling of the face, tongue, or throat, may be avoided by the CAR-T cells or CAR-NK cells based therapy described herein.

The present invention therefore provides means for reducing the side effects of cytokine therapy, and the concomitant use of cytokines with immunotherapies, by enabling local (or locally confined) tumor-specific effects, achieved preferably by systemic administration of the cells, but exerted in a tissue specific manner via cell therapy using CAR-T cells or CAR-NK cells that comprise and express said cytokines and checkpoint inhibitor under the appropriate tissue-specific conditions.

The present invention therefore relates to a genetically modified cells for use as a medicament as described herein, wherein the exogenous nucleic acid comprises a region encoding one or more immune stimulating cytokines operably linked to one or more promoters or promoter/enhancer combinations, wherein the cytokines are selected from the group consisting of IL-15, IL-7, IL-12, IL-2, IL-21, IFN gamma and IFN beta.

The invention also relates to genetically modified cells comprising an exogenous nucleic acid molecule, wherein said exogenous nucleic acid molecule comprises a region encoding a CAR, an immune stimulatory molecule(s) that induce T cell proliferation and/or differentiation (and/or maturation to memory cells and avoidance of tumor-mediated immunosuppression), and a checkpoint inhibitory molecule that inhibit a immunosuppressive milieu, both operably linked to a promoter or promoter/enhancer combination.

The immune stimulatory molecule that induces T cell proliferation and/or differentiation may be a cytokine as described herein or combination of cytokines and checkpoint inhibitory molecule. A combination may be preferred to ensure that solid or liquid tumor cells are attracted appropriately by cytokines, and are directed toward a memory phenotype.

Surprisingly, the CAR-T cells modified with transgenic immune-stimulatory cytokines and checkpoint inhibitory molecule as described herein, show unexpectedly good expression and secretion of said cytokines and checkpoint inhibitors. A skilled person would not expect that these particular cytokines and checkpoint inhibitors could be expressed in sufficient quantities and exported from the cells in sufficient quantities to induce or enhance the desired local immune response, based on either the innate response or and immunotherapy.

The recombinant nucleic acid expression construct described herein, wherein the third nucleic acid sequence region encoding the immune stimulatory cytokine is operably linked to one or more constitutive promoters, preferably a human promotor, more preferably an NFAT promotor.

In one preferred embodiment, the human promotor is an NF-kB promotor.

The use of a “tumor-specific” promoter, or promoter preferentially expressed or induced under inflammatory or “cancer-like” conditions, may show a synergistic effect in combination with the CAR-T cell or CAR-NK cell recruitment signal with respect to reduction of unwanted systemic effect. The CAR-T cells or CAR-NK of the present invention migrate towards inflammatory, in particular tumor, tissue, thereby providing effective means for avoiding systemic expression of the encoded cytokine or checkpoint inhibitor in the body of a patient. The use of a promoter for the expression of the cytokine that is preferentially expressed under conditions of inflammation or of being present in tumor tissue further enhances the reduction in systemic expression in a synergistic manner, thereby providing surprising benefits in the T cell or NK cell-based mode of administration of the cytokines described herein.

In one preferred embodiment, the recombinant nucleic acid expression construct comprises additionally one or more nucleic acid sequences that encode an immune response-stimulating cytokine as described herein.

In one embodiment, the immune response-stimulating cytokine comprises:
A signal sequence, preferably according to SEQ ID NO 29, or a sequence with at least 80% sequence identity to SEQ ID NO 29;
A N-terminal IL15RA polypeptide, preferably according to SEQ ID NO 30, or a sequence with at least 80% sequence identity to SEQ ID NO 30;
A linking loop sequence, preferably according to SEQ ID NO 31, or a sequence with at least 80% sequence identity to SEQ ID NO 31; and
An IL-15 polypeptide, preferably according to SEQ ID NO 32, or a sequence with at least 80% sequence identity to SEQ ID NO 32.

In any of the nucleic acids described herein, the promotor region comprises a nucleotide sequence that induces expression of (a) upon immune effector cell activation. In one embodiment, the constitutive promotor region comprises a promoter of a gene that is induced upon immune effector cell activation. In one embodiment, the constitutive promotor region comprises a nuclear factor of activated T cells (NFAT) promoter, an NF-kB promoter, an IL-15 promoter, or an IL-15 receptor (IL-15) promoter. In one embodiment, the activation-conditional control region comprises one or more binding sites for a transcription modulator, e.g., a transcription factor that induces gene expression upon immune effector cell activation. In one embodiment, the activation-conditional promotor region comprises one or more NFAT binding sides.

The immune response-stimulating cytokine or parts thereof described herein also encompass a sequence with at least 70%, 80%, preferably 90%, or 95%, sequence identity to those sequences disclosed explicitly or disclosed through a sequence formula.

In preferred embodiments, the sequence variants with 70% or more sequence identity to the immune response-stimulating cytokine sequences listed herein maintain IL-15 agonistic activity with essentially the same or similar functional properties of immune response stimulation binding with the specific sequences recited herein, i.e. the PD-1 binding is essentially the same or similar with respect to affinity, specificity and binding mode.

In one preferred embodiment, the recombinant nucleic acid expression construct comprising one or more nucleic acid sequences that encode a CAR comprising:
a CAR signal sequence, preferably according to SEQ ID NO 14, or a sequence with at least 80% sequence identity to SEQ ID NO 14;
an antigen-binding domain of a CAR that specifically recognizes CD44v6, preferably according to SEQ ID NO 15 and SEQ ID NO 19, or a sequence with at least 80% sequence identity to SEQ ID NO 15 and 19;
an immunoglobulin heavy chain extracellular constant region of a CAR, preferably according to SEQ ID NO 23, or a sequence with at least 80% sequence identity to SEQ ID NO 23; and
a CD28 signalling domain, preferably according to SEQ ID NO 24, or a sequence with at least 80% sequence identity to SEQ ID NO 24; wherein the CD28 signaling domain comprises a transmembrane domain, preferably according to SEQ ID NO 25, or a sequence with at least 80% sequence identity to SEQ ID NO 25; and
a CD3 zeta signaling domain, preferably according to SEQ ID NO 26, or a sequence with at least 80% sequence identity to SEQ ID NO 26.

In preferred embodiments, the sequence variants with 70% or more sequence identity to the specific CAR sequences of SEQ ID 15-23 maintain CD44v6 binding with essentially the same or similar functional properties as VH and VL domains with the specific CAR sequences of SEQ ID NO 15-22, i.e. the CD44v6 recognition is essentially the same or similar with respect to affinity, specificity and epitope binding mode.

Furthermore, the order of the light and heavy chain fragments may be inverted upon the desired configuration of the antigen binding fragment.

Additionally, in some embodiments the linker sequence between heavy and light chains can be modified, using routine skills.

Additionally, the nucleic acid sequence encoding the CAR has been codon-optimized in order to improve expression of the CAR. These modifications enable sufficient surface expression on T cells or NK cells and still maintain proper antigen binding. High affinity and high avidity enable CAR-T cells or CAR-NK cells to i) recognize, ii) be activated against, and kill tumor target cells with high, intermediate or low CD44v6 surface expression.

The anti-CD44v6 CAR-T cells and the anti-CD44v6 CAR-NK cells product described herein are characterized by unique properties.

The anti-CD44v6 CAR as described herein has a high affinity and confers high specificity and avidity to T cells and/or NK cells. These properties enable CAR-T cells or CAR-NK cells to i) recognize, ii) be activated against, and iii) kill tumor target cells with high and low CD44v6 surface expression.

The number of CD44v6 antigens expressed on the surfaces of tumor cells can be quantified by using an anti-CD44v6 antibody coupled to a fluorescent-dye in conjunction with Quantibrite beads (from BD). The preferred method applied to quantify CD44v6 antigens expressed on the surfaces of tumor cells is “fluorescence activated cell sorting/cell analysis” (FACS). Fluorescence intensity of beads correlates exactly with the numbers of fluorescent antibodies bound to cells, and this is a measure for the number of CD44v6 molecules on cells.

The VH and VL fragments described herein may be arranged in multiple configurations in the CAR and still maintain high specificity and high affinity for the target epitope. In some embodiments, the CAR may be configured in the VH-VL or VL-VH configuration, with variation in the linker, hinge, transmembrane domain, co-stimulatory domain and/or activation domains, and still maintain its efficacy. This surprising feature of the invention enables greater flexibility in the design of CARs directed against CD44v6, thereby enabling further modification and/or optimization of the CAR structure on the basis of the VH and VL domains described herein, if any further development should be necessary or desired.

The CARs or parts thereof described herein also encompass a sequence with at least 70%, 80%, preferably 90%, sequence identity to those humanized sequences disclosed explicitly or disclosed through a sequence formula.

In preferred embodiments, the sequence variants with 70% or more sequence identity to the specific VH and VL sequences listed herein maintain CD44v6 recognition with essentially the same or similar functional properties as VH and VL domains with the specific sequences recited herein, i.e. the CD44v6 recognition is essentially the same or similar with respect to affinity, specificity and epitope binding mode.

In further embodiments, the invention relates to a chimeric antigen receptor (CAR) polypeptide that comprises one or more linker, spacer, transmembrane, and signaling domains. In one embodiment, the CAR comprises an intracellular domain, which comprises a co-stimulatory domain and a signaling (activation) domain.

The exchange of signaling domains meets the demands for either a strong and rapid effector phase (CD28 co-stimulatory domain), or a long-lasting relapse control as secured by T cell memory population (4-1BB signaling domain). As demonstrated herein, the various signaling domains may be exchanged in multiple configuration, providing a CAR with flexibility with respect to its design without loss of the advantageous binding properties.

Due to the variants (by adding alternative components) employed as the linker, spacer, transmembrane and intracellular domains, it becomes apparent that the various components may be exchanged at required by the skilled person, and the CD44v6 recognizing properties may be maintained, thereby maintaining the desired biological effects.

In some embodiments, CD3 zeta can be absent, particularly in the context of an immune cell lacking CD3 zeta expression.

In one preferred embodiment, the recombinant nucleic acid expression construct additionally comprises one or more nucleic acid sequences encoding a checkpoint inhibitory molecule said a checkpoint inhibitory molecule comprising:
a dominant negative truncated form of a checkpoint protein, preferably a dominant negative truncated PD1 according to SEQ ID NO 28, or a sequence with at least 80% sequence identity to SEQ ID NO 28,
wherein said checkpoint protein is positioned adjacently to a polypeptide cleavage site for cleaving the checkpoint inhibitory molecule from the CAR polypeptide, said cleavage site preferably selected from the group consisting of P2A, T2A, E2A and F2A.

In a preferred embodiment, the recombinant nucleic acid expression construct additionally comprises one or more nucleic acid sequences that encode a checkpoint inhibitory molecule described herein, said a checkpoint inhibitory molecule comprising:
a dominant negative truncated PD1 according to SEQ ID NO 28 or a sequence with at least 80% sequence identity to SEQ ID NO 28,
wherein said checkpoint protein is positioned adjacently to the polypeptide cleavage site P2A for cleaving the checkpoint inhibitory molecule from the CAR polypeptide.

In a preferred embodiment, the recombinant nucleic acid expression construct comprises nucleic acid sequence regions encoding:
a CAR specifically recognizing human CD44v6,
a Checkpoint inhibitory molecule dominant negative truncated PD1 polypeptide,
an immune stimulatory cytokine comprising a signal sequence, a N-terminal IL15RA polypeptide, a linking loop sequence, and an IL-15 polypeptide, wherein the immune stimulatory cytokine is operably linked to one or more promoters, and
a polypeptide cleavage site P2A.

In one embodiment, the immune stimulatory cytokine comprises an immune stimulatory cytokine as described herein, comprising IL-15 peptide and/or IL-15 RA peptide, a signal sequence, and a linking loop sequence. Preferred sequences are disclosed herein.

In one embodiment, the immune stimulatory cytokine comprises an immune stimulatory cytokine as described herein, comprising IL-15 peptide and/or IL-15 RA peptide. Preferred sequences are disclosed herein.

Normally, cells that are potentially cancerous are destroyed by the immune system. All cancer cells undergo changes that differentiate them from their neighbors, the most obvious change being the ability to multiply without inhibition. Cancer cells utilize mechanisms that avoid regular immune system control. Checkpoint proteins have been shown to function by communicating to the immune system that a potentially cancerous cell is not to be destroyed. There may be other molecules signaling that the cell is cancerous, but if there are enough checkpoint proteins on the cell surface, the immune system may overlook cancerous signals.

The checkpoint inhibitory molecule or parts thereof described herein also encompass a sequence with at least 70%, 80%, preferably 90%, sequence identity to those humanized sequences disclosed explicitly or disclosed through a sequence formula.

In preferred embodiments, the sequence variants with 70% or more sequence identity to the dominant negative truncated PD1 sequences listed herein maintain PD-1 binding with essentially the same or similar functional properties PD-1 protein binding with the specific sequences recited herein, i.e. the PD-1 binding is essentially the same or similar with respect to affinity, specificity and binding mode.

A ligand-receptor interaction that has been investigated as a target for cancer treatment is the interaction between the transmembrane programmed cell death 1 protein (PD-1; also known as CD279) and its ligand, PD-1 ligand 1 (PD-L1). In normal physiology PD-L1 on the surface of cells binds to PD1 on the surface of an immune cell, which inhibits the activity of the immune cell. It appears that up-regulation of PD-L1 on the cancer cell surface may allow them to evade the host immune system by inhibiting T cells that might otherwise attack the tumor cell. Antibodies that bind to either PD-1 or PD-L1 and therefore block the interaction may allow the T cells to attack the tumor. In a preferred embodiment, the checkpoint inhibitory molecule is a dominant negative truncated PD-1 polypeptide that blocks PD-1 by binding to PD-1.

Checkpoint inhibitors (also known as immune checkpoint modulators, or CPMs) are designed to lessen the effectiveness of checkpoint proteins. They may have a variety of mechanisms of action, but if effective, they enable the immune system to recognize other molecules on the surface of the cancer cells.

The combined administration of the CAR-T cells expressing an immune stimulating cytokine that induces T cell proliferation and/or differentiation together with a checkpoint inhibitor leads to a synergistic effect with respect to the desired anti-cancer effect. The cytokine or other immune stimulator provides local enhancement of the T cell response against the cancer tissue, whilst the checkpoint inhibitor also enables the T cells to more effectively attack and destroy cancerous tissue. The effects of these two agents are combined in a synergistic manner, resulting in a technical effect greater than the sum of these two aspects when considered alone.

For the use of “off-the-shelf” allogeneic or autologous CAR-T cells or CAR-NK cells, preferably CAR-NK cells, the inventors developed a method of engineering CD44v6 expressing CAR-T cells or CAR-NK cells that are less allogeneic than CAR-expressing cells to date.

In preferred embodiments, the genetically modified cells are allogeneic cells with respect to a patient into which said cells are delivered.

In preferred embodiments, the genetically modified cells are allogeneic T-cells, NK-cells or Treg cells with respect to a patient into which said cells are delivered

In preferred embodiments, the genetically modified cells are autologous cells with respect to a patient into which said cells are delivered.

In preferred embodiments, the genetically modified cells are autologous NK cells with respect to a patient into which said cells are delivered. Particularly advantageous in the present invention is the exchangeable antigen-binding region, while retaining the CAR fragment that comprises the transmembrane region and the co-stimulatory region of the CAR, and which contains additional regions for the expression of immunostimulatory proteins and a checkpoint inhibitor, as described herein.

Thus, the inventors provide a CAR construct backbone useful as a platform construct for engineering any given CAR directed to any given specific antigen.

The invention therefore relates to a method for preparing an enhanced CAR, directed against any given antigen, by essentially modifying the nucleic acid encoding the CAR-backbone to replace the antigen binding domain with an alternative sequence encoding an alternative antigen-binding domain.

In some embodiments, the invention therefore relates to a method for generating and/or modifying a recombinant nucleic acid expression construct as described herein, comprising replacing a nucleic acid sequence encoding an extracellular antigen-binding domain, from within the nucleic acid sequence region encoding a CAR, with a different nucleic acid sequence encoding an extracellular antigen-binding domain with a distinct antigen specificity, using a recombinant nucleic acid technique

The method therefore enables a “cut-and-paste” approach towards generating improved CAR constructs. In some embodiments, the CAR-backbone therefore refers to the CAR-encoding sequence comprising a first nucleic acid sequence region encoding a chimeric antigen receptor (CAR), a second nucleic acid sequence region encoding a checkpoint inhibitory molecule, and a third nucleic acid sequence region encoding an immune stimulatory cytokine. The first nucleic acid sequence region encoding a chimeric antigen receptor (CAR) can therefore be easily manipulated to replace the antigen-binding domain sequence with an alternative sequence. The remaining components of the construct, including for example the second and third nucleic acid sequences, can remain unmodified. In some embodiments, the region encoding the antigen binding domain, or the entire CAR-encoding region can be easily exchanged by established recombinant nucleic acid techniques, in order to modify the specificity of the CAR but maintain the additional improved properties of the combined expression of a checkpoint inhibitory molecule, and an immune stimulatory cytokine.

In another embodiment, the CAR-T cells or CAR-NK cells additionally comprise one or more immune suppression defeating proteins and/or an inducible suicide gene that provokes CAR-T cells or CAR-NK cells death allowing their selective destruction. In some cases, it may be desirable to provide a safety mechanism that allows selective deletion of the administered T cells, as the manipulated T cells can spread and persist for years after administration. Therefore, the method of the invention in some embodiments may include the transformation of the T cells with a recombinant suicide gene. This recombinant suicide gene is used to reduce the risk of direct toxicity and/or uncontrolled proliferation of these T cells after administration to a subject. Suicide genes enable the selective deletion of transformed cells in vivo. In particular, the suicide gene has the ability to convert a non-toxic prodrug into a cytotoxic drug or to express the toxic gene expression product. In other words, “suicide gene” is preferably a nucleic acid that codes for a product, whereby the product itself or in the presence of other compounds causes cell death. In one embodiment, the suicide gene is the herpes simplex virus thymidine kinase.

A further aspect of the invention relates to a chimeric antigen receptor (CAR) polypeptide encoded by the recombinant nucleic acid expression construct as described herein.

In a preferred embodiment, the CAR polypeptide encoded by the recombinant nucleic acid expression construct comprises a CAR signal sequence, an antigen-binding domain of a CAR specifically recognizing CD44v6, an immunoglobulin heavy chain extracellular constant region of a CAR, a CD28 signaling domain, and a CD3 zeta signaling domain.

In a preferred embodiment, the CD3 zeta signaling domain can be absent in the CAR polypeptide, particularly in the context of an immune cell lacking CD3 zeta expression.

In a preferred embodiment, the recombinant nucleic acid expression construct encoding the CAR additionally comprises polypeptides, comprising:
an immune stimulatory cytokine IL-15 polypeptide, comprising a signal sequence, a N-terminal IL-15RA polypeptide, a linking loop sequence, and an IL-15 polypeptide sequence, and
a checkpoint inhibitory molecule dominant negative truncated PD1 polypeptide positioned adjacently to the polypeptide cleavage site P2A for cleaving the checkpoint inhibitory molecule from the CAR polypeptide.

In one embodiment, the immune stimulatory cytokine comprises an immune stimulatory cytokine as described herein, comprising IL-15 peptide and/or IL-15 RA peptide, a signal sequence, and a linking loop sequence. Preferred sequences are disclosed herein.

In one embodiment, the immune stimulatory cytokine comprises an immune stimulatory cytokine as described herein, comprising IL-15 peptide and/or IL-15 RA peptide. Preferred sequences are disclosed herein.

The exchange of signaling domains meets the demands for either a strong and rapid effector phase (CD28 co-stimulatory domain), or a long-lasting relapse control as secured by a T cell memory population (4-1BB signaling domain). As demonstrated herein, the various signaling domains may be exchanged in multiple configuration, providing a CAR with flexibility with respect to its design without loss of the advantageous binding properties.

Due to the variants (by adding alternative components) employed as the linker, spacer, transmembrane and intracellular domains, it becomes apparent that the various components may be exchanged at required by the skilled person, and the CD44v6 recognizing properties may be maintained, thereby maintaining the desired biological effects.

In preferred embodiments, the expression system, preferably in form of a vector, such as a viral vector, plasmid, or a transposon vector, preferably a sleeping beauty vector, an unusual high transduction rate for human T cells can be achieved. The transduction system is variable due to a modular design of the CAR construct, meaning that lentiviruses, adeno-associated viral vector as well as transposons can be employed, depending on the needs and preferences of the skilled person when carrying out the invention. In a preferred embodiment, the adeno-associated viral vector or the lentiviral vectors of the invention is used for the gene transfer into cells, preferably proliferating immune cells, resting immune cells and for gene therapy applications.

In a further aspect of the invention, the invention relates to an isolated nucleic acid molecule, preferably in the form of a vector, such as a viral vector or a transposon vector, preferably a sleeping beauty vector, selected from the group consisting of:
a) nucleic acid molecule comprising a nucleotide sequence
   which encodes a chimeric antigen receptor (CAR) polypeptide according to any embodiment of the CAR described herein,
   which encodes an extracellular antigen-binding domain, a transmembrane domain, and an intracellular domain,
   wherein the extracellular antigen-binding domain is encoded by at least one sequence of SEQ ID NO 2, 3, and/or
b) a nucleic acid molecule comprising a nucleotide sequence
   which encodes a checkpoint inhibitory molecule, according to any embodiment of the CAR described herein, wherein the extracellular checkpoint inhibitory molecule is encoded by at least one sequence of SEQ ID NO 7 or 13, and/or
   which encodes an immune stimulatory cytokine, wherein the immune stimulatory cytokine is encoded by at least one sequence of SEQ ID NO 10 to 12 or 13,
c) a nucleic acid molecule which is complementary to a nucleotide sequence in accordance with a) and b);
d) a nucleic acid molecule comprising a nucleotide sequence having sufficient sequence identity to be functionally analogous/equivalent to a nucleotide sequence according to a) or b) or c), comprising preferably a sequence identity to a nucleotide sequence according to a) or b) or c) of at least 70%;
e) a nucleic acid molecule which, as a consequence of the genetic code, is degenerate to a nucleotide sequence according to a) through d); and/or
f) a nucleic acid molecule according to a nucleotide sequence of a) through e) which is modified by deletions, additions, substitutions, translocations, inversions and/or insertions and is functionally analogous/equivalent to a nucleotide sequence according to a) through c).

The term degenerate to (or degenerated into) refers to differences in nucleotide sequence of a nucleic acid molecule, but according to the genetic code, do not lead to differences in amino acid protein product of the nucleotide sequence after translation.

The invention further relates to a method for producing a genetically modified cell, comprising delivering or transferring a nucleic acid construct according encoding the CAR as described herein, said method can be employed with one or more gene transfer techniques including a lentiviral vector, a retroviral vector, an adenoviral vector, an adeno-associated viral vector, an alphavirus vector, a chemical transfection, an electroporation, and a mRNA transfection, preferably the adeno-associated viral vector.

Currently, adeno-associated virus (AAV) vectors are recognised as the gene transfer vectors providing safest and most efficient profile for gene transfer in vivo. Several AAV serotypes, including AAV2 and AAV8, have been used to target human cells effectively, and long-term expression of the therapeutic transgene has been recorded. The group of AAV serotypes include, among others, the serotypes AAV1, AAV2, AAV3, AAV4, AAV5, AAV6, AAV7, AAV8, AAV9, AAV10, AAV11 and RhlO. Transfer of the genetic information/nucleic acid molecule for the CD44v6 CAR also includes CRISPR/Cas and TALEN mediated insertion into target cell lines, preferably T lymphocytes, Natural Killer cells, and induced pluripotent stem cells, iPS. In one embodiment, the iPS cell line is ND50039. All suitable methods for transferring the genetic information/nucleic acid molecule for the CD44v6 CAR into the cell expressing said CAR are encompassed by the present invention, and a suitable method may be selected by a skilled person when carrying out the invention. For example, multiple methods of transforming T cells are known in the art, including any given viral-based gene transfer method, such as those based on modified Retroviridae, and non-viral methods such as DNA-based transposons and direct transfer of DNA or RNA by electroporation. Suitable methods for transferring the genetic information/nucleic acid molecule into any cell type using chemical transfection is well known by skilled person. In one embodiment, the genetic information/nucleic acid molecule is transferred into PS cells, preferably the PS cell line ND50039, by electroporation.

Additionally, the signaling components of the CAR construct have been exchanged in a three step cloning procedure that allows for a modular composition, and tailor-made construction by a skilled person, of clinically applicable anti-CD44v6 CARs.

A further aspect of the invention relates to a genetically modified cell, comprising a recombinant nucleic acid expression construct or a CAR, wherein the cells are preferably a human immune cell, more preferably selected from the group consisting of induced pluripotent stem cells (iPSC), preferably iPSC line ND50039, immortalized immune cells including NK-92 and YT cell, primary immune cells including a natural killer (NK) cell, a cytokine-induced killer cell (CIK), T lymphocytes wherein the T lymphocytes are preferably a CD4 or CD8 T cell, more preferably a cytotoxic T lymphocyte or a T helper cell or tumor infiltrating lymphocytes (TIL).

A further aspect of the invention relates to genetically modified cells, comprising a recombinant nucleic acid expression construct or a CAR described herein, wherein the cells are iPSC line ND50039.

Immune cells are preferably selected from T cells, CD4+ T cell, CD8+ T cell, B cells, dendritic cells, granulocytes, innate lymphoid cells (ILCs), megakaryocytes, monocytes/macrophages, Natural Killer (NK) cells, NK-92, YT cells, MCF-7, Jurkat cells, MC32A cells, HEK293 cells, platelets, red blood cells (RBCs) and/or thymocytes.

Adoptive cell transfer uses T cell-based cytotoxic responses to attack cancer cells. T cells that have a natural or genetically engineered reactivity to a patient's cancer are generated in vitro and then transferred back into the cancer patient. Autologous tumor-infiltrating lymphocytes have been used as an effective treatment for patients with metastatic melanoma. This can be achieved by taking T cells that are found with the tumor of the patient, which are trained to attack the cancerous cells. These T cells may be referred to as tumor-infiltrating lymphocytes (TIL). Such T cells may be stimulated to multiply in vitro using high concentrations of IL-2, anti-CD3 and allo-reactive feeder cells. Traditionally, these T cells are then transferred back into the patient along with exogenous administration of IL-2 to further boost their anti-cancer activity.

The present invention therefore encompasses adoptive cell transfer in combination with administration of the CAR-T cells described herein. It is encompassed within the invention that administration of genetically modified T cells prior to immune cells (e.g. CAR-Ts) will enhance the chemo-attraction of CAR-Ts and other immune effector cells administered during adoptive cell transfer due to the expression of appropriate chemokines. The expression of stimulating cytokines will enhance the activation of T cells only locally, preferably within or in proximity to the tumor, and subsequently lead to a memory effector cell phenotype, thereby prolonging the therapeutic effect of the treatment.

The cells may however be obtained from a subject distinct from the intended patient, therefore being considered allogenic. As used herein, cells are “allogenic” with respect to a subject if it or any of its precursor cells are from another subject of the same species. As used herein, cells are “autologous” with respect to a subject if it or its precursor cells are from the same subject. In a preferred embodiment, the immune cells are autologous to the subject of medical treatment.

In a preferred embodiment the genetically modified immune cells comprising a nucleic acid molecule or vector as described herein, and/or expressing a CAR as described herein, is characterized in that it is a CD4+ and/or CD8+ T cell, preferably a mixture of CD4+ and CD8+ T cells. These T cell populations, and preferably the composition comprising both CD4+ and CD8+ transformed cells, show particularly effective cytolytic activity against various solid or liquid tumors, such as colorectal cancer, preferably against those cells and/or the associated medical conditions described herein.

In a preferred embodiment, the genetically modified immune cells comprising a nucleic acid molecule or vector as described herein, and/or expressing a CAR as described herein, are CD4+ and CD8+ T cells, preferably in a ration of 1:10 to 10:1, more preferably in a ratio of 5:1 to 1:5, 2:1 to 1:2 or 1:1. Administration of CD44v6-directed modified CAR-T cells expressing the CAR described herein at the ratios mentioned, preferably at a 1:1 CD4+/CD8+ ratio, lead to beneficial characteristics during treatment of the diseases mentioned herein, for example these ratios lead to improvedtherapeutic response and reduced toxicity.

An additional and surprising aspect of the invention is an improved stability of the CAR as disclosed herein. The CAR polypeptide can readily be stored for extended periods under appropriate conditions without any loss of binding affinity.

In one embodiment the immune cells are derived from peripheral human blood, human cord blood or induced pluripotent stem cells (iPSC). In one embodiment, the immune cells are derived from the iPS cell line ND50039.

In one embodiment, the genetically modified immune cells are derived from iPSC that have been genetically modified with the recombinant nucleic acid construct described herein before differentiation into immune cells.

In one embodiment, the genetically modified immune cells are derived from induced pluripotent stem cell line ND50039 that have been genetically modified with the recombinant nucleic acid construct described herein before differentiation into immune cells.

In one embodiment, genetically modified immune cells are derived from iPS cells or iPSC line ND50039 genetically modified by electroporation or chemical transduction with the recombinant nucleic acid expression construct described herein, comprising:
a CAR as described herein specifically recognizing human CD44v6,
a checkpoint inhibitory molecule dominant negative truncated PD1 polypeptide as described herein,
an immune stimulatory cytokine as described herein, comprising IL-15 peptide and/or IL-15 RA peptide, a signal sequence, and a linking loop sequence, and
a polypeptide cleavage site P2A,
wherein said genetically modified iPS cells or iPSC line ND50039 are differentiated into NK cells or T-cells.

Transplantation of autologous or allogeneic cells loaded with the CAR transgene described herein is feasible. For example, when reintroduced back to patients after autologous cell transplantation, the T cells modified with the CAR of the invention as described herein may recognize and kill tumor cells. CIK cells may have enhanced cytotoxic activity compared to other T cells, and therefore represent a preferred embodiment of an immune cells of the present invention. As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into CAR-T effector cells in vivo or in vitro. Progenitors can be iPS cells that become immune effector cells under defined culture conditions.

In one embodiment, the progenitor iPS cell line ND50039 is cultured under defined culture conditions to become immune effector cells.

In one embodiment, the immune response-stimulating cytokine maintains or enhances the activity, survival and/or number of immune cells within and/or in proximity to tumor tissue.

The present invention encompasses adoptive cell transfer in combination with administration of the CAR-T cells described herein. It is encompassed within the invention that administration of genetically modified CAR-T cells prior to immune cells (e.g. CAR-Ts) will enhance the chemoattraction of CAR-Ts and other immune effector cells administered during adoptive cell transfer due to the expression of appropriate chemokines. The expression of stimulating cytokines will enhance the activation of T cells only locally, preferably within or in proximity to the tumor, and subsequently lead to a memory effector cell phenotype, thereby prolonging the therapeutic effect of the treatment.

The present invention further comprises adoptive cell transfer in in combination with administration of the CAR-T cells described herein. It is encompassed within the invention that administration of genetically modified CAR-NK cells will trigger lysing of tumor-transformed cells in a major histocompatibility class I or II independent manner. NK cells are capable of directly lysing tumor-transformed cells and can also act as bridge between the innate and adaptive immune responses to enhance recognition and destruction of tumors by adaptive immune cells. Distinct from the mechanism by which T-cells lyse tumor cells, which requires recognition of tumor antigens presented in the context of major histocompatibility class I or II by a specific T-cell receptor, NK cells are able to kill tumor cells without prior sensitization to tumor antigens. NK cells act as a first line of defense against newly transformed cells. NK cells kill tumor targets through receptor-mediated cytotoxicity. This process is dependent on the presence of tumor specific antibodies bound to tumor surface antigens.

This feature of the CAR-NK cell product allows to reduce risks for a graft versus host disease in an allogeneic cell context. The CAR-NK cells product can be preferably used as allogeneic and “off the shelf” product. In the meaning of the invention, the CAR construct without the antigen binding domain is particularly suitable as a platform technology with a flexibly exchangeable antigen recognition region.

In one embodiment, the genetically modified cells shall be used as a medicament in the treatment of a medical disorder associated with the presence of pathogenic cells expressing CD44v6, preferably cancer cells, more preferably cancer stem cells of solid or liquid malignancies, more preferably cancer cells in colon cancer, rectal cancer, lung cancer, breast cancer, liver cancer, pancreatic cancer, leukemias, acute myeloid leukemia, multiple myeloma, malignant lymphomas, stomach cancer, and ovarian cancer, more preferably metastatic tumor cells positive for CD44v6. Treatment with the genetically modified cells according to the invention can in some embodiments be combined with one or more anti-cancer treatments or medicaments, preferably selected from the group of antibody therapy, vaccines, oncolytic viral therapy, chemotherapy, radiation therapy, cytokine therapy, dendritic cell therapy, gene therapy, hormone therapy, laser light therapy, immune suppression or transplantation.

The invention relates further to methods of treatment of the medical conditions described herein, comprising typically the administration of a therapeutically effective amount of the CAR, or immune cells expressing said CAR, to a patient in need of said treatment.

The present invention provides the technical solution for an efficient solid or liquid tumor treatment using the CAR construct described herein with an antigen binding domain that selectively recognizes tumor specific antigens, actively stimulates immune cells, especially T cells and NK cells, and encourages an immune stimulating tumor microenvironment by an efficient checkpoint inhibitor.

The invention further relates to a pharmaceutical composition comprising a genetically modified cell according the inventions described herein and a pharmaceutically acceptable carrier. The composition described herein may be administered to a patient subcutaneously, intradermally, intratumorally, intranodally, intramedullary, intramuscularly, by intravenous or intralymphatic injection, or intraperitoneally.

In one embodiment, the therapeutic cell product of the pharmaceutical composition is for use in the treatment and/or prevention of a medical disorder described herein.

In one embodiment, the pharmaceutical composition can be administered to patients before, after and/or in combination with one or more anti-cancer treatments or medicaments including antibody therapy, vaccines, oncolytic viral therapy, chemotherapy, radiation therapy, cytokine therapy, dendritic cell therapy, gene therapy, hormone therapy, laser light therapy, B-cell ablative therapy, T-cell ablative therapy immune suppression, peripheral blood stem cell transplantation or bone marrow stem cell transplantation.

In a preferred embodiment, the genetically modified cells according to the invention or a cell line derived from the genetically modified cells may be used to treat, modify or prevent disorders associated to cancer, cancer metastasis and/or autoimmunity, preferably disorders associated with the presence of pathogenic cells expressing CD44v6.

### DETAILED DESCRIPTION OF THE INVENTION

An important function of the immune system is to recognize and eliminate tumors. Tumor antigens are either specifically expressed on tumor cells and not found on non-pathogenic cells or abnormally expressed, e.g. at least twice above the level found in non-pathogenic cells. Antigens, specifically found on tumor cells, may appear foreign to the immune system and their presence may cause the immune cells to attack the transformed tumor cells. Some antigens are derived from oncogenic viruses such as the human papilloma virus, which causes cervical cancer. One example of an abnormally expressed protein is an enzyme called tyrosinase which, when expressed in high amounts, converts certain skin cells (e.g. melanocytes) into tumors called melanomas. Another possible source of tumor antigens are proteins that are normally important for the regulation of cell growth and survival and that mutate into molecules called oncogenes, which often cause cancer. However, a native immune response often fails to eliminate tumors and a therapeutic invention is urgently needed.

The main response of the immune system to tumors is to destroy the abnormal cells with the help of killer T cells, sometimes with helper T cells. Tumor antigens are presented on MHC class I molecules in a similar way to viral antigens. This enables killer T cells to recognize the tumor cell as abnormal. NK cells also kill tumor cells in a similar way, especially if the tumor cells have fewer MHC class I molecules on their surface than normal; this is a common phenomenon in tumors. Some tumor cells also release products that inhibit the immune response, for example by secreting the cytokine TGF-β, which suppresses the activity of macrophages and lymphocytes. Cytokine-induced killer cells (CIK) are a group of immune effector cells that exhibit a hybrid T- and NK-like phenotype. They are produced by ex vivo incubation of mononuclear cells from human peripheral blood (PBMC) or cord blood with interferon-gamma (IFN-γ), anti-CD3 antibody, recombinant human interleukin (IL-) 1 and recombinant human interleukin (IL)-2.

The present invention therefore provides means to enable and/or enhance an anti-tumor immune response by simultaneous expression of a chimeric antigen receptor (CAR) directed against a tumor antigen, e.g. CD44v6, a checkpoint inhibitory molecule, and an immune-stimulatory cytokine, e.g. IL-15, in a genetically modified immune cell, e.g. T lymphocyte, cytokine-induced killer cells (CIK), NK cell, as described herein.

Immunotherapy is to be understood in the context of the present invention to encompass any therapeutic agent that uses the immune system to treat cancer. Immunotherapy exploits the fact that cancer cells have subtly different molecules on their surface that can be detected by the immune system. These molecules, known as cancer antigens, are most commonly proteins, but also include molecules such as carbohydrates, lipids, and lipoproteins. Immunotherapy provokes or enhances the immune system in attacking the tumor cells by using these antigens as targets. Further, the present invention exceptionally combines the CAR described herein with an immune stimulatory cytokine to induce activation and proliferation of T and/or natural killer (NK) cells and with a checkpoint inhibitory molecule to enhance the endogenous anti-tumor activity of the immune system.

Immunotherapy encompasses, without limitation, cellular and antibody therapy. Cellular therapies typically involve the administration of immune cells isolated from the blood or from a tumor of the patient. Immune cells directed towards the tumor to be treated are activated, cultured and returned to the patient where the immune cells attack the cancer. Cell types that can be used in this way are, without limitation, natural killer cells, lymphokine-activated killer cells, cytotoxic T cells, and dendritic cells. Dendritic cell therapy provokes anti-tumor responses by causing dendritic cells to present tumor antigens. Dendritic cells present antigens to lymphocytes, which activates them, priming them to kill other cells that present the antigen.

Antibodies are proteins produced by the immune system that bind to a target antigen on the cell surface. Those that bind to cancer antigens may be used to treat cancer. Cell surface receptors are common targets for antibody therapies and include for example CD19, CD44, CD20, CD274, and CD279. Once bound to a cancer antigen, antibodies can induce antibody-dependent cell-mediated cytotoxicity, activate the complement system, or prevent a receptor from interacting with its ligand, all of which can lead to cell death. Multiple antibodies are approved to treat cancer, including Alemtuzumab, Ipilimumab, Nivolumab, Ofatumumab, and Rituximab.

Antibody-dependent cell-mediated cytotoxicity (ADCC) is a mechanism of attack by the immune system that requires antibodies to bind to target cell surfaces. Antibodies are formed of a binding region (Fab) and the Fc region that can be detected by immune cells via their Fc surface receptors. Fc receptors are found on many immune system cells, including natural killer cells. When natural killer cells encounter antibody-coated cells, the latter's Fc regions interact with their Fc receptors, leading to the release of perforin and granzyme B. These two chemicals programmed cell death (apoptosis) in the tumor cell. Effective antibodies include Rituximab, Ofatumumab, and Alemtuzumab.

The complement system includes blood proteins that can cause cell death after an antibody binds to the cell surface. Generally, the system deals with foreign pathogens, but can be activated with therapeutic antibodies in cancer. The system can be triggered if the antibody is chimeric, humanized or human; as long as it contains the IgG1 Fc region. Complement can lead to cell death by activation of the membrane attack complex, known as complement-dependent cytotoxicity; enhancement of antibody-dependent cell-mediated cytotoxicity; and CR3-dependent cellular cytotoxicity. Complement-dependent cytotoxicity occurs when antibodies bind to the cancer cell surface, the C1 complex binds to these antibodies and subsequently protein pores are formed in the cancer cell membrane.

The CAR of the present invention is capable of enabling and/or enhancing the immunotherapies described herein through their unique properties derived through the combination with immune-stimulating transgene cytokine and the endogenous anti-tumor activity boosting checkpoint inhibitory molecule.

Chimeric Antigen Receptors:

According to the present invention, a chimeric antigen receptor (CAR), comprises an extracellular antigen-binding domain, comprising an antibody or antibody fragment that binds a target antigen, a transmembrane domain, and an intracellular domain. CARs are typically described as comprising an extracellular ectodomain (antigen-binding domain) derived from an antibody and an endodomain comprising signaling modules derived from T cell signaling proteins.

In a preferred embodiment, the ectodomain preferably comprises variable regions from the heavy and light chains of an immunoglobulin configured as a single-chain variable fragment (scFv). The scFv is preferably attached to a hinge region that provides flexibility and transduces signals through an anchoring transmembrane moiety to an intracellular signaling domain. The transmembrane domains originate preferably from either CD8a or CD28. In the first generation of CARs the signaling domain consists of the zeta chain of the TCR complex. The term “generation” refers to the structure of the intracellular signaling domains. Second generation CARs are equipped with a single costimulatory domain originated from CD28 or 4-1 BB. Third generation CARs already include two costimulatory domains, e.g. CD28, 4-1 BB, ICOS or OX40, CD3 zeta. The present invention preferably relates to a second or third generation CAR.

In various embodiments, genetically engineered receptors that redirect cytotoxicity of immune effector cells toward B cells are provided. These genetically engineered receptors referred to herein as chimeric antigen receptors (CARs). CARs are molecules that combine antibody-based specificity for a desired antigen (e.g., CD44v6) with a T cell receptor-activating intracellular domain to generate a chimeric protein that exhibits a specific anti-CD44v6 cellular immune activity. As used herein, the term, “chimeric,” describes being composed of parts of different proteins or DNAs from different origins.

CARs contemplated herein, comprise an extracellular domain (also referred to as a binding domain or antigen-binding domain) that binds to CD44v6, a transmembrane domain, and an intracellular domain, or intracellular signaling domain. Engagement of the anti-CD44v6 antigen binding domain of the CAR with CD44v6 on the surface of a target cell results in clustering of the CAR and delivers an activation stimulus to the CAR-containing cell. The main characteristic of CARs are their ability to redirect immune effector cell specificity, thereby triggering proliferation, cytokine production, phagocytosis or production of molecules that can mediate cell death of the target antigen expressing cells in a major histocompatibility complex (MHC) independent manner, exploiting the cell specific targeting abilities of monoclonal antibodies, soluble ligands or cell specific co-receptors.

In various embodiments, a CAR comprises an extracellular binding domain that comprises a humanized CD44v6-specific binding domain; a transmembrane domain; one or more intracellular signaling domains. In particular embodiments, a CAR comprises an extracellular binding domain that comprises a humanized anti-CD44v6 antigen binding fragment thereof; one or more spacer domains; a transmembrane domain; one or more intracellular signaling domains. The “extracellular antigen-binding domain” or “extracellular binding domain” are used interchangeably and provide a CAR with the ability to specifically bind to the target antigen of interest, CD44v6. The binding domain may be derived either from a natural, synthetic, semisynthetic, or recombinant source. Preferred are scFv domains.

“Specific binding” is to be understood as via one skilled in the art, whereby the skilled person is clearly aware of various experimental procedures that can be used to test binding and binding specificity. Methods for determining equilibrium association or equilibrium dissociation constants are known in the art. Some cross-reaction or background binding may be inevitable in many protein-protein interactions; this is not to detract from the “specificity” of the binding between CAR and epitope. “Specific binding” describes binding of an anti-CD44v6 antibody or antigen binding fragment thereof (or a CAR comprising the same) to CD44v6 at greater binding affinity than background binding. The term “directed against” is also applicable when considering the term “specificity” in understanding the interaction between antibody and epitope.

An “antigen (Ag)” refers to a compound, composition, or substance that can stimulate the production of antibodies or a T cell response in an animal. In particular embodiments, the target antigen is an epitope of a CD44v6 polypeptide. An “epitope” refers to the region of an antigen to which a binding agent binds. Epitopes can be formed both from contiguous amino acids or noncontiguous amino acids juxtaposed by tertiary folding of a protein.

“Single-chain Fv” or “scFv” antibody fragments comprise the VH and VL domains of an antibody, wherein these domains are present in a single polypeptide chain and in either orientation {e.g., VL-VH or VH-VL). Generally, the scFv polypeptide further comprises a polypeptide linker between the VH and VL domains which enables the scFv to form the desired structure for antigen binding. In preferred embodiments, a CAR contemplated herein comprises antigen-specific binding domain that is a scFv and may be a murine, human or humanized scFv. Single chain antibodies may be cloned from the V region genes of a hybridoma specific for a desired target.

In particular embodiments, the antigen-specific binding domain that is a humanized scFv that binds a human CD44v6 polypeptide. An illustrative example of a variable heavy chain that is suitable for constructing anti-CD44v6 CARs contemplated herein include, but are not limited to the amino acid sequence set forth in SEQ ID NO: 19. An illustrative example of a variable light chain that is suitable for constructing anti-CD44v6 CARs contemplated herein include, but is not limited to the amino acid sequence set forth in SEQ ID NO: 15.

Antibodies and Antibody Fragments:

The CAR comprises an extracellular antigen-binding domain, comprising preferably an antibody or antibody fragment that binds CD44v6 polypeptide. Antibodies or antibody fragments of the invention therefore include, but are not limited to polyclonal, monoclonal, bispecific, human, humanized or chimeric antibodies, single chain fragments (scFv), single variable fragments (ssFv), single domain antibodies (such as VHH fragments from nanobodies), Fab fragments, F(ab′)2 fragments, fragments produced by a Fab expression library, anti-idiotypic antibodies and epitope-binding fragments or combinations thereof of any of the above, provided that they retain similar binding properties of the CAR described herein, preferably comprising the corresponding CDRs, or VH and VL regions as described herein. Also, mini-antibodies and multivalent antibodies such as diabodies, triabodies, tetravalent antibodies and peptabodies can be used in a method of the invention. The immunoglobulin molecules of the invention can be of any class (i.e. IgG, IgE, IgM, IgD and IgA) or subclass of immunoglobulin molecules. Thus, the term antibody, as used herein, also includes antibodies and antibody fragments comprised by the CAR of the invention, either produced by the modification of whole antibodies or synthesized de novo using recombinant DNA methodologies.

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 1 10 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins.

The CARs of the invention are intended to bind against mammalian, in particular human, protein targets. The use of protein names may correspond to either mouse or human versions of a protein.

Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore.

Humanized antibodies comprising one or more CDRs of antibodies of the invention or one or more CDRs derived from said antibodies can be made using any methods known in the art. For example, four general steps may be used to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; 5,225,539; 6,548,640.

The term humanized antibody means that at least a portion of the framework regions, and optionally a portion of CDR regions or other regions involved in binding, of an immunoglobulin is derived from or adjusted to human immunoglobulin sequences. The humanized, chimeric or partially humanized versions of the mouse monoclonal antibodies can, for example, be made by means of recombinant DNA technology, departing from the mouse and/or human genomic DNA sequences coding for H and L chains or from cDNA clones coding for H and L chains. Humanized forms of mouse antibodies can be generated by linking the CDR regions of non-human antibodies to human constant regions by recombinant DNA techniques (Queen et al., 1989; WO 90/07861). Alternatively, the monoclonal antibodies used in the method of the invention may be human monoclonal antibodies. Human antibodies can be obtained, for example, using phage-display methods (WO 91/17271; WO92/01047).

As used herein, humanized antibodies refer also to forms of non-human (e.g. murine, camel, llama, shark) antibodies that are specific chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin.

As used herein, human or humanized antibody or antibody fragment means an antibody having an amino acid sequence corresponding to that of an antibody produced by a human and/or has been made using any of the techniques for making human antibodies known in the art or disclosed herein. Human antibodies or fragments thereof can be selected by competitive binding experiments, or otherwise, to have the same epitope specificity as a particular mouse antibody. The humanized antibodies of the present invention surprisingly share the useful functional properties of the mouse antibodies to a large extent. Human polyclonal antibodies can also be provided in the form of serum from humans immunized with an immunogenic agent. Optionally, such polyclonal antibodies can be concentrated by affinity purification using amyloid fibrillar and/or non-fibrillar polypeptides or fragments thereof as an affinity reagent. Monoclonal antibodies can be obtained from serum according to the technique described in WO 99/60846.

Variable Regions and CDRs

A variable region of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies.

There are a number of techniques available for determining CDRs, such as an approach based on cross-species sequence variability (i.e., Kabat et al. Sequences of Proteins of Immunological Interest, (5th ed., 1991, National Institutes of Health, Bethesda Md.)); and an approach based on crystallographic studies of antigen-antibody complexes (AI-Lazikani et al. (1997) J. Molec. Biol. 273:927-948). Alternative approaches include the IMGT international ImMunoGeneTics information system, (Marie-Paule Lefranc). The Kabat definition is based on sequence variability and is the most commonly used method. The Chothia definition is based on the location of the structural loop regions, wherein the AbM definition is a compromise between the two used by Oxford Molecular's AbM antibody modelling software (refer www.bioinf.org.uk: Dr. Andrew C. R. Martin's Group). As used herein, a CDR may refer to CDRs defined by one or more approach, or by a combination of these approaches.

In some embodiments, the invention provides an antibody or fragment thereof incorporated into a CAR, wherein said antibody or fragment thereof comprises at least one CDR, at least two, at least three, or more CDRs that are substantially identical to at least one CDR, at least two, at least three, or more CDRs of the antibody of the invention. Other embodiments include antibodies which have at least two, three, four, five, or six CDR(s) that are substantially identical to at least two, three, four, five or six CDRs of the antibodies of the invention or derived from the antibodies of the invention. In some embodiments, the at least one, two, three, four, five, or six CDR(s) are at least about 70%, 75%, 85%, 86%, 87%, 88%, 89%, 90%, 95%, 96%, 97%, 98%, or 99% identical to at least one, two or three CDRs of the antibody of the invention. It is understood that, for purposes of this invention, binding specificity and/or overall activity is generally retained, although the extent of activity may vary compared to said antibody (may be greater or lesser).

Additional Components of the CAR

In certain embodiments, the CARs contemplated herein may comprise linker residues between the various domains, added for appropriate spacing and conformation of the molecule, for example a linker comprising an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. Illustrative examples of linkers include glycine polymers; glycine-serine polymers; glycine-alanine polymers; alanine-serine polymers; and other flexible linkers known in the art, such as the Whitlow linker. Glycine and glycine-serine polymers are relatively unstructured, and therefore may be able to serve as a neutral tether between domains of fusion proteins such as the CARs described herein. In particular embodiments, the binding domain of the CAR is followed by one or more “spacers” or “spacer polypeptides,” which refers to the region that moves the antigen binding domain away from the effector cell surface to enable proper cell/cell contact, antigen binding and activation. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain comprises the CH2 and CH3 domains of IgG1 or IgG4. In one embodiment the Fc-binding domain of such a spacer/hinge region is mutated in a manner that prevents binding of the CAR to Fc-receptors expressed on macrophages and other innate immune cells.

The binding domain of the CAR may in some embodiments be followed by one or more “hinge domains,” which play a role in positioning the antigen binding domain away from the effector cell surface to enable proper cell/cell contact, antigen binding and activation. A CAR may comprise one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered. In another embodiment, the hinge domain comprises a PD1, CD 152, or CD8 alpha hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from the alpha, beta or zeta chain of the T cell receptor, CD3s, O′Ω3ξ, CD4, CD5, CD8 alpha, CD9, CD16, CD22, CD27, CD28, CD33, CD37, CD45, CD64, CD80, CD86, CD134, CD137, CD152, CD154, and PD1. In one embodiment, the CARs contemplated herein comprise a TM domain derived from CD8 alpha or CD28.

In particular embodiments, CARs contemplated herein comprise an intracellular signaling domain. An “intracellular signaling domain,” refers to the part of a CAR that participates in transducing the message of effective anti-CD44v6 CAR binding to a human CD44v6 polypeptide into the interior of the immune effector cells to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cells to perform a specialized function. CARs contemplated herein comprise one or more co-stimulatory signaling domains to enhance the efficacy, expansion and/or memory formation of T cells expressing CAR receptors. As used herein, the term, “co-stimulatory signaling domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to an antigen. The costimulatory molecule is a cell surface molecule other than an antigen receptor or its ligand that contributes to an efficient immune response. Costimulatory molecules include MHC class I molecules, BTLA and Toll ligand receptors, OX40, CD27, CD28, CDS, ICAM-1, LFA-1 (CD11a/CD18), ICOS (CD278) and 4-1 BB (CD137). Including, but not limited to, further examples of such costimulatory molecules are CDS, ICAM-1, GITR, BAFFR, HVEM (LIGHTTR), SLAMF7, NKp80 (KLRF1), NKp44, NKp30, NKp46, CD160, CD19, CD4, CD8 alpha, CD8 beta, IL2Rbeta, IL2Rgamma, IL7Ralpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD 26), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a and ligands that specifically bind to CD83.

A costimulatory intracellular signaling domain can be the intracellular portion of a costimulatory molecule. In one embodiment, the CAR comprises an intracellular domain, which comprises a co-stimulatory domain and a signalling (activation) domain. The CAR construct may therefore include an intracellular signaling domain (CD3 zeta) of the native T cell receptor complex and one or more co-stimulatory domains that provide a second signal to stimulate full T cell activation. Co-stimulatory domains are thought to increase CAR-T cell cytokine production and facilitate T cell replication and T cell persistence. Co-stimulatory domains have also been shown to potentially prevent CAR-T cell exhaustion, increase T cell anti-tumor activity, and enhance survival of CAR-T cells in patients. As a non-limiting example, CAR constructs with the 4-1 BB co-stimulatory domain have been associated with gradual, sustained expansion and effector function, increased persistence, and enriched central memory cells (TCM) in the T cell subset composition in preclinical studies. 4-1 BB is a member of the tumor necrosis factor (TNF) superfamily, and it is an inducible glycoprotein receptor in vivo that is primarily expressed on antigen-activated CD4 and CD8 T cells. As a non-limiting example, CD28 is member of the immunoglobulin (Ig) superfamily. It is constitutively expressed on resting and activated CD4 and CD8 T cells and plays a critical role in T cell activation by stimulating the PI3K-AKT signal transduction pathway. In one embodiment, the intracellular domain comprises both 4-1 BB and CD28 co-stimulatory domains. Other co-stimulatory domains comprise ICOS and OX40 that can be combined with the CD3 zeta signaling (activation) domain.

The cytokines described herein may relate to any mammalian cytokine corresponding to the cytokine named herein. Preferably, the cytokines relate to the human cytokines, or mouse cytokines. Cancer immunotherapy attempts to stimulate the immune system to reject and destroy tumors. Initially, immunotherapy treatments involved administration of cytokines such as “Interleukin”, as described herein.

Checkpoint inhibitors, also known as immune checkpoint modulators, are designed to lessen the effectiveness of checkpoint proteins. They may have a variety of mechanisms of action, but if effective, they enable the immune system to recognize other molecules on the surface of the cancer cells. The checkpoint inhibitor of the immune response is selected from the group consisting of: PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta. Polypeptides

“Peptide” “polypeptide”, “polypeptide fragment” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring.

In various embodiments, the CAR polypeptides contemplated herein comprise a signal (or leader) sequence at the N-terminal end of the protein, which co-translationally or post-translationally directs transfer of the protein. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as disclosed herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cells that has been obtained from an in vivo tissue or organ and is substantially free of extracellular matrix.

Nucleic Acids

As used herein, the terms “polynucleotide” or “nucleic acid molecule” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100%) sequence identity to any of the reference sequences described herein, typically where the variant maintains at least one biological activity of the reference sequence. In various illustrative embodiments, the present invention contemplates, in part, polynucleotides comprising expression vectors, viral vectors, and transfer plasmids, and compositions, and cells comprising the same. Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and animal viruses. Examples of categories of animal viruses useful as vectors include, without limitation, retrovirus (including lentivirus), adenovirus, adeno-associated virus, herpesvirus {e.g., herpes simplex virus), poxvirus, baculovirus, papillomavirus, and papovavirus {e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, the coding sequences of the chimeric proteins disclosed herein can be ligated into such expression vectors for the expression of the chimeric protein in mammalian cells. The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

Vectors

In particular embodiments, cells (e.g., an immune effector cell, such as a T cell) is transduced with an adeno-associated viral vector, a retroviral vector, e.g., a lentiviral vector, encoding a CAR. For example, an immune effector cells is transduced with a vector encoding a CAR that comprises a humanized anti-CD44v6 antibody or antigen binding fragment that binds a CD44v6 polypeptide, with a transmembrane and intracellular signaling domain, such that these transduced cells can elicit a CAR-mediated cytotoxic response.

In some embodiments, a particular advantage of the invention is the use of AAVs for the genetic transfer of the recombinant nucleic acid construct of the present invention due to its high safety and transduction efficiency in vivo. Variants of AAV, such as AAV and capsid variants, can provide or transfer polynucleotides and/or proteins that offer desired or therapeutic benefits and thereby treat various diseases. For example, AAV1, AAV2, AAV3, AAV4, AAV5, AAV6, AAV7, AAV8, AAV9, AAV10, AAV11, Rh10, Rh74 or AAV-2i8 and variants thereof and AAV capsid variants (e.g. 4-1) are a useful vector for providing a therapeutic gene to treat cells, tissues and organs. Recombinant viruses and AAV vectors of the invention containing the vector genome (virus or AAV) (encapside and encapsidate) contain additional factors which function in cis or trans. The AAV vector is selected from a group including AAV1, AAV2, AAV3, AAV4, AAV5, AAV6, AAV7, AAV8, AAV9, AAV10, AAV11, Rh10, Rh74 or AAV-2i8 AAV capsid sequences, or AAV1, AAV2, Capsid variants of AAV3, AAV4, AAV5, AAV6, AAV7, AAV8, AAV9, AAV10, AAV11, Rh10, Rh74 or AAV-2i8 are included.

Retroviruses are a common tool for gene delivery. In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lenti virus. As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide comprising a CAR to a cell.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In further embodiments of the invention, CrispR/Cas and TALEN-mediated insertion of the CD44v6 CAR encoding nucleic acid may be employed. Appropriate vectors for CrispR/Cas and TALEN-mediated insertion are known to a skilled person.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of cells or to a viral particle that mediates nucleic acid transfer. Viral particles will typically include various viral components and sometimes also host cell components in addition to nucleic acid(s).

The term viral vector may refer either to a virus or viral particle capable of transferring a nucleic acid into a cells or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus.

In a preferred embodiment the invention therefore relates to a method for transfecting cells with an expression vector encoding a CAR. For example, in some embodiments, the vector comprises additional sequences, such as sequences that facilitate expression of the CAR, such a promoter, enhancer, poly-A signal, and/or one or more introns. In preferred embodiments, the CAR-coding sequence is flanked by transposon sequences, such that the presence of a transposase allows the coding sequence to integrate into the genome of the transfected cell.

In a preferred embodiment the invention therefore relates to a method for transfecting cells with an expression vector encoding a CAR using electroporation. Electroporation is a physical method, which creates pores in the cell membrane by applying an electric shock to the cell. These pores allow the increased diffusion of materials into the cell. This increased permeability allows for easier transfection.

Sonoporation is similar to electroporation except it uses ultrasound to stimulate the cell membrane. The ultrasound also creates turbulence in the fluid surrounding the cell, which increases the rate of diffusion across the membrane.

In a preferred embodiment the invention therefore relates to a method for transfecting cells with an expression vector encoding a CAR using chemical transfection. Chemical transfection refers to the calcium phosphate transfection which is well known by skilled persons. Calcium phosphate transfection uses calcium phosphate bonded to DNA (A Watson and D Latchman, “Gene Delivery into Neuronal Cells by Calcium Phosphate-Mediated Transfection”, Methods, Volume 10, Issue 3, December 1996, Pages 289-291). It has been suggested to use calcium phosphate particles as agents for transfection of therapeutic polynucleotides in gene therapy. See U.S. Pat. No. 5,460,831. DNA or RNA is attached to the particulate core and delivered to a target cell, resulting in expression of therapeutic proteins.

In some embodiments, the genetically transformed cells are further transfected with a transposase that facilitates integration of a CAR coding sequence into the genome of the transfected cells. In some embodiments the transposase is provided as DNA expression vector. However, in preferred embodiments, the transposase is provided as an expressible RNA or a protein such that long-term expression of the transposase does not occur in the transgenic cells. For example, in some embodiments, the transposase is provided as an mRNA (e.g., an mRNA comprising a cap and poly-A tail). Any transposase system may be used in accordance with the embodiments of the present invention. However, in some embodiments, the transposase is salmonid-type Tel-like transposase (SB). For example, the transposase can be the so called “Sleeping beauty” transposase, see e.g., U.S. Pat. No. 6,489,458, incorporated herein by reference. In some embodiments, the transposase is an engineered enzyme with increased enzymatic activity. Some specific examples of transposases include, without limitation, SB 10, SB 1 1 or SB 100× transposase (see, e.g., Mates et al, 2009, Nat Genet. 41 (6):753-61, or U.S. Pat. No. 9,228,180, herein incorporated by reference). For example, a method can involve electroporation of cells with an mRNA encoding an SB 10, SB 1 1 or SB 100× transposase.

Sequence Variants

Sequence variants of the claimed nucleic acids, proteins, antibodies, antibody fragments and/or CARs, for example those defined by % sequence identity, that maintain similar binding properties of the invention are also included in the scope of the invention. Such variants, which show alternative sequences, but maintain essentially the same binding properties, such as target specificity, as the specific sequences provided are known as functional analogs, or as functionally analogous.

Sequence identity relates to the percentage of identical nucleotides or amino acids when carrying out a sequence alignment.

The recitation “sequence identity” as used herein refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. Substitutions may be carried out that preferably do not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, lie and Leu; the non-polar aromatic amino acids Phe, Trp and Tyr; the neutral polar amino acids Ser, Thr, Cys, Gin, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

Substitution variants have at least one amino acid residue in the antibody molecule removed and a different residue inserted in its place. The sites of greatest interest for substitutional mutagenesis include the hypervariable regions, but framework alterations are also contemplated. If such substitutions result in a change in biological activity, then more substantial changes, denominated “exemplary substitutions” in the table immediately below, or as further described below in reference to amino acid classes, may be introduced and the products screened.

Potential Amino Acid Substitutions:

| | Preferred | |
|---|---|---|
| Original | conservative | |
| residue | substitutions | Examples of exemplary substitutions |
| Ala (A) | Val | Val; Leu; Ile |
| Asg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn, Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogs; citrulline and methionine sulfoxide which are neutral non-polar analogs, phenylglycine which is an aromatic neutral analog; cysteic acid which is a negatively charged analog and ornithine which is a positively charged amino acid analog. Like the naturally occurring amino acids, this list is not exhaustive, but merely exemplary of the substitutions that are well known in the art.

Genetically Modified Cells and Immune Cells

The present invention contemplates, in particular embodiments, cells genetically modified to express the CARs contemplated herein, for use in the treatment of medical conditions. As used herein, the term “genetically engineered” or “genetically modified” refers to the addition of extra genetic material in the form of DNA or RNA into the total genetic material in a cell. The terms, “genetically modified cells”, “genetically modified immune cells”, “modified cells,” and, “redirected cells,” are used interchangeably. As used herein, the term “gene therapy” refers to the introduction-permanently or transiently—of extra genetic material in the form of DNA or RNA into the total genetic material in a cell that restores, corrects, or modifies expression of a gene, or for the purpose of expressing a therapeutic polypeptide, e.g., a CAR. In particular embodiments, the CARs contemplated herein are introduced and expressed in immune effector cells so as to redirect their specificity to a target antigen of interest, e.g., a CD44v6 polypeptide.

An “immune cell” or “immune effector cell” are any cells of the immune system that has one or more effector functions (e.g., cytotoxic cell killing activity, secretion of cytokines, induction of ADCC and/or CDC). Immune effector cells can be also differentiated from iPSCs (induced pluriotent stem cells) or derived from peripheral human blood and/or human cord blood.

iPSC cell lines can also be ordered from public vendors and cell repositories, such as the NINDS Human Cell and Data Repository (https://stemcells.nindsqenetics.org/). For example, publically available iPCS cells can be selected from a group of iPS cell line NDS00159; NDS00249; NDS00250, NDS00251, NDS00252, NDS00253, NDS00254, NDS00255, NDS00256, NDS00257, NDS00258, NDS00259, NDS00260, NDS00261, NDS00262, NDS00263, ND50039. In one embodiment, the iPS cell line NDS00159 is used.

Immune effector cells of the invention can be autologous/autogeneic (“self) or non-autologous (“non-self,” e.g., allogeneic, syngeneic or xenogeneic). “Autologous”, as used herein, refers to cells from the same subject, and represent a preferred embodiment of the invention. “Allogeneic,” as used herein, refers to cells of the same species that differ genetically to the cells in comparison.

“Syngeneic,” as used herein, refers to cells of a different subject that are genetically identical to the cells in comparison. “Xenogeneic,” as used herein, refers to cells of a different species to the cells in comparison. In preferred embodiments, the cells of the invention are autologous or allogeneic. Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes, cytokine-induced killer cells (CIK cells) or activated T lymphocytes. Cytokine-induced killer (CIK) cells are typically CD3- and CD56-positive, non-major histocompatibility complex (MHC)-restricted, natural killer (NK)-like T lymphocytes. T cells can be a T helper (Th; CD4+ T cell) cell, for example a T helper 1 (Th1) or a T helper 2 (Th2) cell. T cells can be cytotoxic T cells (CTL; CD8+ T cell), CD4+CD8+ T cell, CD4 CD8 T cell, or any other subset of T cells.

“Immortalized”, as used herein, refers to immortalized cells as a population of cells that would not normally reproduce for an unlimited number of cell cycles. Due to a mutation, immortal cells escape from a normal cellular senescence and instead keep undergoing the cell proliferation. The mutation can occur spontaneously or induced by UV light, genetic manipulation, or entry by a virus, toxin or bacteria into the cell. The cells can therefore be cultivated in vitro over a longer period of time for experimental, therapeutic or medical purposes. Said immortalized immune cells include K13, αβ T cells, γδ T cells, NK cells, NKT cells, NK-92 and YT cells, stem cells, or stem cell-derived cells including cells of the aforementioned immune system, preferably NKT cells, NK-92 cells, YT cells, and NK cells. An immortalized T cell line may retains its lytic function.

Other illustrative populations of T cells suitable for use in particular embodiments include naive T cells and memory T cells and stem cell-like memory cells TSCM).

For example, when reintroduced back to patients after autologous cell transplantation, the T cells modified with the CAR of the invention as described herein may recognize and kill tumor cells. CIK cells may have enhanced cytotoxic activity compared to other T cells, and therefore represent a preferred embodiment of an immune cells of the present invention.

As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Progenitors can be iPSCs that become immune effector cells under defined culture conditions. The present invention provides methods for making the immune effector cells which express the CAR contemplated herein. In one embodiment, the method comprises transfecting or transducing immune effector cells isolated from an individual such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In particular embodiments, prior to in vitro manipulation or genetic modification of the immune effector cells described herein, the source of cells is obtained from a subject. In particular embodiments, the CAR-modified immune effector cells comprise T cells. T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation, antibody-conjugated bead-based methods such as MACS™ separation (Miltenyi). In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flow through centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells can be further isolated by positive or negative selection techniques. One method for use herein is cell sorting and/or selection via negative magnetic immunoadherence or flow cytometry that uses a cocktail of monoclonal antibodies directed to cell surface markers present on the cells negatively selected.

PBMC may be directly genetically modified to express CARs using methods contemplated herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion. CD8+ cells can be obtained by using standard methods. In some embodiments, CD8+ cells are further sorted into naive, central memory, and effector cells by identifying cell surface antigens that are associated with each of those types of CD8+ cells.

In some embodiments, the immune cells of the present invention, for example the T cells or NK cells described herein, can be obtained from inducible pluripotent stem cells (iPSCs) using methods known to a skilled person. Accepted approaches for producing CAR-T cells rely on the genetic modification and expansion of mature circulating T cells. Such processes utilize autologous T cells and reduce risk of graft-versus-host (GvHD) disease from allogeneic T cells through endogenous TCR expression as well as rejection through MHC incompatibility. As an alternative, direct in vitro differentiation of engineered T cells from pluripotent stem cells, such as inducible pluripotent stem cells, provides an essentially unlimited source of cells that can be genetically modified to express the CAR of the present invention. In some embodiments, a so-called master iPSC line can be maintained, which represents a renewable source for consistently and repeatedly manufacturing homogeneous cell products. In some embodiments, the transformation of a master iPSC cell line with the CAR encoding nucleic acid is contemplated, prior to expansion and differentiation to the desired immune cell, preferably T cells or NK cells.

In one embodiment, the transformation of the master iPSC cell line ND50039 with the CAR encoding nucleic acid construct is contemplated, before expansion and differentiation to the desired immune cell, preferably T cells or NK cells. T lymphocytes can for example be generated from prior genetically modified iPSCs, such that iPSCs could be modified with the CAR encoding nucleic acids and subsequently expanded and differentiated to T cells for administration to the patient.

NK cells can also be generated from prior genetically modified iPSCs, such that iPSCs could be modified with the CAR encoding nucleic acid constructs and subsequently expanded and differentiated to NK cells for administration to the patient. Differentiation to the appropriate immune cells, such as T cells or NK cells, could also be conducted from the iPSCs before transformation with CAR encoding nucleic acids and expansion, and thus transformation with CAR-encoding nucleic acid constructs and expansion of the appropriate immune cells, such as T cells and NK cells, prior to administration. All possible combinations of iPSC expansion, genetic modification and expansion to provide suitable numbers of cells for administration are contemplated in the invention.

The immune effector cells, such as T cells or NK cells, can be genetically modified following isolation using known methods, or the immune effector cells can be activated and expanded (or differentiated in the case of progenitors) in vitro prior to being genetically modified. In a particular embodiment, the immune effector cells, such as T cells or NK cells, are genetically modified with the chimeric antigen receptors contemplated herein (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

In one embodiment, the invention provides a method of storing genetically modified murine, human or humanized CAR protein expressing immune effector cells which target a CD44v6 protein, comprising cryopreserving the immune effector cells such that the cells remain viable upon thawing. A fraction of the immune effector cells expressing the CAR proteins can be cryopreserved by methods known in the art to provide a permanent source of such cells for the future treatment of patients afflicted with the B cell, T cell, NK cell, dendritic cell (DC), cytotoxic induced killer cells (CIK) related condition. When needed, the cryopreserved transformed immune effector cells can be thawed, grown and expanded for more such cells.

Compositions and Formulations

The compositions contemplated herein may comprise one or more polypeptides, polynucleotides, vectors comprising same, genetically modified immune effector cells, etc., as contemplated herein. Compositions include, but are not limited to, pharmaceutical compositions. A “pharmaceutical composition” refers to a composition formulated in pharmaceutically-acceptable or physiologically-acceptable solutions for administration to a cells or an animal, either alone, or in combination with one or more other modalities of therapy. It will also be understood that, if desired, the compositions of the invention may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

AAV vectors, lentiviral vectors and/or other compositions, agents, drugs, biologics (proteins) can be incorporated into pharmaceutical compositions, eg, pharmaceutically acceptable carriers or excipients. Such pharmaceutical compositions are particularly useful for administration and delivery to a subject in vivo or ex vivo

In particular embodiments, compositions of the present invention comprise an amount of CAR-expressing immune effector cells contemplated herein. As used herein, the term “amount” refers to “an amount effective” or “an effective amount” of a genetically modified therapeutic cell, e.g., T cell, NK cell, CIK cell, to achieve a beneficial or desired prophylactic or therapeutic result, including clinical results.

A “prophylactically effective amount” refers to an amount of a genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount. The term prophylactic does not necessarily refer to a complete prohibition or prevention of a particular medical disorder. The term prophylactic also refers to the reduction of risk of a certain medical disorder occurring or worsening in its symptoms.

A “therapeutically effective amount” of a genetically modified immunce cells may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject {e.g., a patient). When a therapeutic amount is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells or CIK cells or NK cells described herein may be administered at a dosage of 10² to 10¹⁰ cells/kg body weight, preferably 10⁵ to 10⁷ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/mL, generally 10⁸ cells/mL or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells may be administered. CAR expressing cell compositions may be administered multiple times at dosages within these ranges. The cells may be allogeneic, syngeneic, xenogeneic, or autologous to the patient undergoing therapy.

Generally, compositions comprising the cells activated and expanded as described herein may be utilized in the treatment and prevention of diseases that arise in individuals who are immunocompromised. In particular, compositions comprising the CAR-modified T cells contemplated herein are used in the treatment of cancer, preferably solid or liquid, more preferably solid malignancies, more preferably rectal cancer, lung cancer, breast cancer, liver cancer, pancreatic cancer, stomach cancer, and ovarian cancer, more preferably metastatic tumor cells positive for CD44v6. The CAR-modified T cells of the present invention may be administered either alone, or as a pharmaceutical composition in combination with carriers, diluents, excipients, and/or with other components such as interleukins or other immune response stimulating cytokines e.g. IL-15 and/or checkpoint inhibitory molecules, e.g. PD1 polypeptide or a PD1-antibody, or cell populations. In particular embodiments, pharmaceutical compositions contemplated herein comprise an amount of genetically modified T cells, in combination with one or more pharmaceutically or physiologically acceptable carriers, diluents or excipients.

Pharmaceutical compositions of the present invention comprising a CAR-expressing immune effector cell population, such as T cells or NK cells or CIK cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for parenteral administration, e.g., intravascular (intravenous or intraarterial), intraperitoneal or intramuscular administration.

The liquid pharmaceutical compositions, whether they are solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In a particular embodiment, compositions contemplated herein comprise an effective amount of CAR-expressing immune effector cells, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

A combined immunotherapy encompasses simultaneous treatment, co-treatment orjoint treatment, and includes the administration of genetically modified immune cells expressing a nucleic acid construct encoding a CAR combined with immunotherapies, such as checkpoint inhibitors and/or immune stimulatory cytokines, whereby treatment may occur within minutes of each other, in the same hour, on the same day, in the same week or in the same month as one another. A combination medicament, comprising one or more of said genetically modified immune cells with another immunotherapeutic, may also be used in order to co-administer the various components in a single administration or dosage.

As used herein, the term “tumor microenvironment” relates to the cellular environment in which any given tumor exists, including the tumor stroma, surrounding blood vessels, immune cells, fibroblasts, other cells, signalling molecules, and the ECM.

Therapeutic Methods

The genetically modified immune effector cells contemplated herein provide improved methods of adoptive immunotherapy for use in the treatment of medical disorders associated with the presence of pathogenic cells expressing CD44v6 that include, but are not limited to immunoregulatory conditions and hematological and solid malignancies.

As use herein, “medical disorders associated with the presence of pathogenic cells expressing CD44v6” refer to medical conditions, such as a cancer or autoimmune disease, in which the cells involved in pathophysiology of the disease demonstrate expression of CD44v6, and preferably presentation of CD44v6 on the cell surface. The expression of CD44v6 can be determined by various methods known to a skilled person, for example by isolating cells from a patient and assessing these by PCR using primers directed CD44v6 transcripts, immune-staining with anti CD44v6 antibodies, or by analysis by flow cytometry. Such pathogenic cells may typically be pathogenic mature B cells and/or memory B cells and/or pathogenic T cells and/or T follicular helper cells and/or tumor stem cells and/or solid tumor cells and/or liquid tumors and/or metastatic cancer cells and/or NK cells and/or CIK cells, and/or dendritic cells.

“Cancer”, as used herein, is a disease characterized by the uncontrolled growth of abnormal cells. Cancer refers to any type of cancerous growth or carcinogenic process, metastatic tissue or malignant transformed cells, tissues or organs, regardless of histopathological type or invasive stage. Cancer cells can spread locally or to other parts of the body via the bloodstream and lymphatic system. Cancer cells spreading to other parts to the body are termed “metastatic cells” or “metastatic tumor cells”. The terms “tumor” and “cancer”, used herein, are utilized interchangeably, e.g. both terms include solid and liquid, e.g. general or circulating tumors, premalignant and malignant cancers and tumors. Examples of liquid cancers include, but not limited to, acute lymphocytic leukemia (ALL), chronic lymphocytic leukemia (CLL), acute myeloid Leukemia (acute myelogenous leukemia (AML), chronic bone marrow cancer, chronic myelogenous leukemia (CML), Hodgkin lymphoma, non-Hodgkin lymphoma, and myeloma. Examples of solid tumors are liver, lung, breast, lymphatic system, digestive organs (e.g. colon), urogenital organs (e.g. kidney, urothelial cells), prostate and throat, malignant organ systems including tumors such as sarcomas, adenocarcinomas and cancer. Examples for breast cancer that can be treated with the are ductal carcinoma in situ (DCIS), lobular carcinoma in situ (LCIS), invasive ductal carcinoma (IDC), invasive ductal carcinoma including tubular, medullary, mucinous, papillary, and cribriform carcinomas, invasive lobular carcinoma (ILC), inflammatory breast cancer, male breast cancer, Paget's Disease of the nipple, phyllodes tumors of the breast, recurrent and/or metastatic breast cancer. Adenocarcinoma includes most malignant tumors such as colon cancer, rectal cancer, renal cell cancer, liver cancer, non-small cell lung cancer, small intestine cancer and esophageal cancer. In certain forms the cancer is a melanoma, e.g. an advanced stage melanoma. Metastatic lesions of the cancer can also be treated or prevented with the methods and compositions of the invention. Examples of other types of cancer that can be treated are bone cancer, pancreatic cancer, skin cancer, head and neck cancer, skin or intraocular melanoma, uterine cancer, ovarian cancer, rectal cancer, anal cancer, stomach cancer, testicular cancer, faropius duct cancer, Endometrial cancer, cervical cancer, vaginal cancer, vulvar cancer, Hodgkin's disease, non-Hodgkin's lymphoma, esophageal cancer, small intestine cancer, endocrine cancer, thyroid cancer, parathyroid cancer, adrenal cancer, soft tissue sarcoma, urethral cancer, penile cancer, acute myeloid leukemia, chronic myelocytic leukaemia, acute lymphoblastic leukaemia, chronic or acute leukaemia including chronic lymphatic leukaemia, solid tumor in childhood, lymphatic lymphoma, bladder cancer, kidney or ureter cancer, renal pelvis cancer, neoplasm of the central nervous system (CNS) primary CNS lymphoma, tumor angiogenesis, spinal axis tumor, brainstem glioma, pituitary adenoma, Kaposi's sarcoma, epidermoid carcinoma, squamous cell carcinoma, asbestos-induced T cell lymphoma Including a combination of environmental cancer and cancer including things Treatment of metastatic cancer, e.g. metastatic cancer that expresses PD-L1 (lwai et al. (2005) Int. Immunol. 17: 133-144) can be performed with the inhibitory molecules described in this invention.

“Cancer-associated antigen” or “tumor antigen” is expressed interchangeably on the surface of cancer cells, either completely or as a fragment (e.g. MHC/peptide), and the drug targets cancer cells preferentially. Molecules (usually proteins, carbohydrates or lipids) that are useful for. Tumor antigen refers to an antigen that is commonly found in a specific hyperproliferative disease. In one aspect, the antigen of hyperproliferative disease of the present invention is a primary or metastatic melanoma, thymoma, lymphoma, sarcoma, lung cancer, liver cancer, non-Hodgkin's lymphoma, Hodgkin's lymphoma, Leukemia, uterine cancer, cervical cancer, bladder cancer, derived from cancers such as kidney and breast cancer, prostate cancer, ovarian cancer, adenocarcinoma such as pancreatic cancer and the like. In certain embodiments, the tumor antigen is a marker expressed on both normal and cancer cells, such as a marker of cell lineage, like CD19 on B cells. In certain embodiments, the tumor antigen is overexpressed in cancer cells compared to normal cells, e.g. 1-fold overexpression, 2-fold overexpression, 3-fold overexpression or more overexpression compared to normal cells. Molecules of the cell surface. In certain forms, a tumor antigen is a cell surface molecule that is inadequately synthesised in cancer cells, e.g. a molecule that contains deletions, additions or mutations compared to a molecule expressed in normal cells. In certain embodiments, the tumor antigen is expressed exclusively, completely or as a fragment (e.g. MHC/peptide) on the cell surface of cancer cells and is not synthesised or expressed on the surface of normal cells. In certain embodiments, a CAR of the invention comprises a CAR containing an antigen binding domain (e.g. an antibody or antibody fragment) which binds to an MHC-presenting peptide. Normally, peptides derived from endogenous proteins fill the pockets of major histocompatibility complex (MHC) class I molecules and are recognized by the T cell receptor (TCR) on CD8+T lymphocytes. Class I MHC complexes are constitutively expressed by all nucleated cells. In cancer, virus- and/or tumor-specific peptides/MHC complexes represent a unique class of cell surface targets for immunotherapy. TCR-like antibodies have been described which target peptides of virus or tumor antigens associated with human leukocyte antigen (HLA)-A1 or HLA-A2 (see e.g. Sastry et al., J Virol. 2011 85 (5): 1935-1942; Sergeeva et al, Blood, 2011 117 (16): 4262-4272; Verma et al., J Immunol 2010 184 (4): 2156-2165; Willemsen et al., Gene Ther 2001 8 (21): 1601-1608; Dao et al., Sci Transl Med 2013 5 (176): 176ra33; Tassev et al., Cancer Gene Ther 2012 19 (2): 84-100). For example, TCR-like antibodies can be identified by searching a library such as a human scFv phage display library.

In particular embodiments, compositions comprising CAR-modified T cells contemplated herein are used in the treatment of cancer, including but not limited to solid or liquid malignancies, such as, for example, rectal cancer, lung cancer, breast cancer, liver cancer, pancreatic cancer, stomach cancer, ovarian cancer, and metastatic tumor cells positive for CD44v6, or hematological malignancies such as, for example, non-Hodgkin's lymphoma (NHL), such as B cells NHL orT cell non-Hodgkin's lymphoma, with orwithout a leukemic tumor cell dissemination.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” or “prophylactic” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In one embodiment, a method of treating a cancer related condition in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

Sequences

Preferred Nucleic Acid Sequences of the Invention:

| SEQ | | | |
|---|---|---|---|
| ID | Info | Specific | Nucleic Acid Sequence |
| 1 | First protein | CAR | ATGGGATGGAGCTGTATCATCCTCTTCCTGGTAGCAACAACAGCCACC |
| | signal | | GGCGTCCATAGC |
| | sequence | | |
| 2 | CAR variable | CD44v6 | GAGATCGTCCTGACTCAGAGCCCTGCAACCCTGTCCCTGTCTCCAGGC |
| | domain | | GAACGGGCTACACTGAGCTGCTCTGCCTCTAGCAGCATTAACTACATTT |
| | | | ACTGGTATCAGCAGAAGCCAGGCCAAGCCCCGAGGCTCCTTATTTACT |
| | | | TGACTTCCAACCTGGCTAGTGGAGTGCCGGCTAGGTTCAGCGGGAGC |
| | | | GGCTCAGGCACAGACTTCACATTGACAATATCAAGCCTGGAACCCGAA |
| | | | GATTTTGCTGTGTACTATTGTCTCCAATGGTCTTCTAATCCTCTCACGTT |
| | | | CGGAGGGGGGACTAAGGTGGAGATTAAGGGGTCAACCTCTGGCAGCG |
| | | | GGAAACCTGGCTCTGGCGAGGGCAGCACCAAGGGTCAAGTCCAGCTC |
| | | | CAAGAAGTGCAACTCGTGGAATCAGGAGGCGGCTTGGTGAAGCCCGG |
| | | | CGGCTCCTTGAGACTGTCTTGCGCTGCTAGCGGGTTTACATTCTCATCT |
| | | | TACGACATGAGTTGGGTTCGCCAGGCACCTGGAAAAGGCCTCGAGTGG |
| | | | GTCAGCACTATTTCTAGCGGCGGATCTTACACCTACTATCTGGACAGCA |
| | | | TCAAAGGACGCTTCACAATTTCTCGCGACAATGCTAAGAACTCTTTGTA |
| | | | TCTCCAGATGAACTCCCTTAGAGCCGAGGATACTGCTGTCTACTATTGC |
| | | | GCTCGCCAGGGACTCGATTACTGGGGACGGGGCACACTCGTGACTGT |
| | | | CTCCTCTGTGCGGCCGCCGAGC |
| 3 | Immunoglobulin | | CCCAAATCTTGTGACAAAACTCACACATGCCCACCGTGCCCAGCACCT |
| | heavy chain | | GAACTCCTGGGGGGACCGTCAGTCTTCCTCTTCCCCCCAAAACCCAAG |
| | extracellular | | GACACCCTCATGATCTCCCGGACCCCTGAGGTCACATGCGTGGTGGTG |
| | constant region | | GACGTGAGCCACGAAGACCCTGAGGTCAAGTTCAACTGGTACGTGGAC |
| | (CAR) | | GGCGTGGAGGTGCATAATGCCAAGACAAAGCCGCGGGAGGAGCAGTA |
| | | | CAACAGCACGTACCGTGTGGTCAGCGTCCTCACCGTCCTGCACCAGGA |
| | | | CTGGCTGAATGGCAAGGAGTACAAGTGCAAGGTCTCCAACAAAGCCCT |
| | | | CCCAGCCCCCATCGAGAAAACCATCTCCAAAGCCAAAGGGCAGCCCCG |
| | | | AGAACCACAGGTGTACACCCTGCCCCCATCCCGGGAGGAAATGACCAA |
| | | | AAACCAGGTCAGCCTGACCTGCCTGGTCAAAGGCCTTCTATCCAGCGA |
| | | | CATCGCCGTGGAGTGGGAGAGCAATGGGCAGCCGGAGAACAACTACA |
| | | | AGACCACGCCTCCCGTGCTGGACTCCGACGGCTCCTTCTTCCTCTACA |
| | | | GCAAGCTCACCGTGGACAAGAGCAGGTGGCAGCAGGGGAACGTCTTC |
| | | | TCATGCTCCGTGATGCATGAGGCTCTGCACAACCACTACACGCAGAAG |
| | | | AGCCTCTCCCTGTCTCCGGGTAAG |
| 4 | Co-stimulatory | CD28 | GATCCCAAACTCTGCTACTTTTGGGTGCTGGTGGTGGTTGGTGGAGTC |
| | signaling | | CTGGCTTGCTATAGCTTGCTAGTAACAGTGGCCTTTATTATTTTCTGGGT |
| | domain (CAR) | | GAGGAGTAAGAGGAGCAGGCTCCTGCACAGTGACTACATGAACATGAC |
| | | | TCCCCGCCGCCCCGGGCCCACCCGCAAGCATTACCAGCCCTATGCCC |
| | | | CACCACGCGACTTCGCAGCCTATCGCTCC |
| 5 | T cell | CD3 zeta | AGAGTGAAGTTCAGCAGGAGCGCAGACGCCCCCGCGTACCAGCAGGG |
| | activation | signaling | CCAGAACCAGCTCTATAACGAGCTCAATCTAGGACGAAGAGAGGAGTA |
| | signaling | domain | CGATGTTTTGGACAAGAGACGTGGCCGGGACCCTGAGATGGGGGGAA |
| | domain (CAR) | (CAR) | AGCCGCAGAGAAGGAAGAACCCTCAGGAAGGCCTGTACAATGAACTGC |
| | | | AGAAAGATAAGATGGCGGAGGCCTACAGTGAGATTGGGATGAAAGGCG |
| | | | AGCGCCGGAGGGGCAAGGGGCACGATGGCCTTTACCAGGGTCTCAGT |
| | | | ACAGCCACCAAGGACACCTACGACGCCCTTCATATGCAGGCTCTGCCA |
| | | | CCTAGC |
| 6 | Recombinant | P2A | GGATCCGGCGCAACAAACTTCTCTCTGCTGAAACAAGCCGGAGATGTC |
| | protein | cleavage | GAAGAGAATCCTGGACCG |
| | cleavage motif | sequence | |
| | sequence | | |
| 7 | Dominant | Dominant | ATGCAGATCCCACAGGCGCCCTGGCCAGTCGTCTGGGCGGTGCTACA |
| | negative | negative | ACTGGGCTGGCGGCCAGGATGGTTCTTAGACTCCCCAGACAGGCCCT |
| | truncated | truncated | GGAACCCCCCCACCTTCTCCCCAGCCCTGCTCGTGGTGACCGAAGGG |
| | Checkpoint | PD1 | GACAACGCCACCTTCACCTGCAGCTTCTCCAACACATCGGAGAGCTTC |
| | inhibitor | | GTGCTAAACTGGTACCGCATGAGCCCCAGCAACCAGACGGACAAGCTG |
| | | | GCCGCCTTCCCCGAGGACCGCAGCCAGCCCGGCCAGGACTGCCGCTT |
| | | | CCGTGTCACACAACTGCCCAACGGGCGTGACTTCCACATGAGCGTTGT |
| | | | CAGGGCCCGGCGCAATGACAGCGGCACCTACCTCTGTGGGGCCATCT |
| | | | CCCTGGCCCCCAAGGCGCAGATCAAAGAGAGCCTGCGGGCAGAGCTC |
| | | | AGGGTGACAGAGAGAAGGGCAGAAGTGCCCACAGCCCACCCCAGCCC |
| | | | CTCACCCAGGCCAGCCGGCCAGTTCCAAACCCTGGTGGTTGGTGTCGT |
| | | | GGGCGGCCTGCTGGGCAGCCTGGTGCTGCTAGTCTGGGTCCTGGCCG |
| | | | TCATCTGCTCCCGGGCCGCACGAGGGTGA |
| 8 | Promotor | NFAT | TAATCCCAGTGTGGTGGTACGGAATTCTCTAGACTGCCGGATCCAAGC |
| | | | TGGAGGAAAAACTGTTTCATACAGAAGGCGTGGAGGAAAAACTGTTTCA |
| | | | TACAGAAGGCGTGGAGGAAAAACTGTTTCATACAGAAGGCGTGGAGGA |
| | | | AAAACTGTTTCATACAGAAGGCGTCGCGAATTCGCGGAGACTCTAGAG |
| | | | GGTATATAATGGAAGCTCGATTTCCAGCTTGGCATTCCGGTACTGTTGG |
| | | | TAAACACCAAGCTTCACC |
| 9 | Second protein | Cytokine | ATGGTGAGCAAGGGCGAGGAGCTGTTCACCGGGGTGGTGATGGCACC |
| | Signal | | AAGAAGGGCTCGCGGATGCCGGACCCTCGGTCTCCCGGCGCTGCTTC |
| | sequence | | TGCTGCTGCTGCTCCGGCCGCCGGCGACGCGGGGC |
| 10 | Cytokine | IL-15RA | ATCACGTGTCCTCCACCCATGTCCGTGGAACACGCAGACATCTGGGTC |
| | subunit | 1-360 bp | AAGAGCTACAGCTTGTACTCCAGGGAGCGGTACATTTGTAACTCTGGTT |
| | | | TCAAGAGGAAAGCCGGCACGTCCAGCCTGACGGAGTGCGTGTTGAAC |
| | | | AAGGCCACGAATGTCGCCCACTGGACAACCCCCAGTCTCAAATGCATT |
| | | | AGAGACCCTGCCCTGGTTCACCAAAGGCCAGCGCCACCC |
| 11 | Linker | Linking | AGCGGCGGAGGATGTGGAGGCGGAGGCTGTGGCGGAAGCGGAGGCG |
| | Sequence | loop | GAGGCAGT |
| | | sequence | |
| 12 | Cytokine | IL-15 | ATCCAGAACTGGGTGAATGTCATAAGTGATCTGAAGAAAATTGAAGATC |
| | | | TTATTCAGTCTATGCATATTGATGCTACTCTATATACTGAAAGTGATGTT |
| | | | CACCCCAGTTGCAAAGTGACAGCAATGAAGTGCTTTCTCTTGGAGCTG |
| | | | CAAGTTATTTCACTTGAGTCCGGAGATGCAAGTATTCATGATACAGTGG |
| | | | AAAATCTGATCATCCTTGCAAACGACAGTTTGTCTTCTAATGGGAATGTA |
| | | | ACAGAATCTGGATGCAAAGAATGTGAGGAACTGGAGGAAAAAAATATTA |
| | | | AAGAATTTTTGCAGAGTTTTGTACATATTGTCCAAATGTTCATCAACACT |
| | | | TCTTGATAA |
| 13 | Full | CD44v6 | ATGGGATGGAGCTGTATCATCCTCTTCCTGGTAGCAACAACAGCCACC |
| | recombinant | CAR with | GGCGTCCATAGCGAGATCGTCCTGACTCAGAGCCCTGCAACCCTGTCC |
| | nucleic acid | Dominant | CTGTCTCCAGGCGAACGGGCTACACTGAGCTGCTCTGCCTCTAGCAGC |
| | expression | negative | ATTAACTACATTTACTGGTATCAGCAGAAGCCAGGCCAAGCCCCGAGG |
| | construct | truncated | CTCCTTATTTACTTGACTTCCAACCTGGCTAGTGGAGTGCCGGCTAGGT |
| | | PD1 and | TCAGCGGGAGCGGCTCAGGCACAGACTTCACATTGACAATATCAAGCC |
| | | IL-15 | TGGAACCCGAAGATTTTGCTGTGTACTATTGTCTCCAATGGTCTTCTAAT |
| | | | CCTCTCACGTTCGGAGGGGGGACTAAGGTGGAGATTAAGGGGTCAAC |
| | | | CTCTGGCAGCGGGAAACCTGGCTCTGGCGAGGGCAGCACCAAGGGTC |
| | | | AAGTCCAGCTCCAAGAAGTGCAACTCGTGGAATCAGGAGGCGGCTTGG |
| | | | TGAAGCCCGGCGGCTCCTTGAGACTGTCTTGCGCTGCTAGCGGGTTTA |
| | | | CATTCTCATCTTACGACATGAGTTGGGTTCGCCAGGCACCTGGAAAAG |
| | | | GCCTCGAGTGGGTCAGCACTATTTCTAGCGGCGGATCTTACACCTACT |
| | | | ATCTGGACAGCATCAAAGGACGCTTCACAATTTCTCGCGACAATGCTAA |
| | | | GAACTCTTTGTATCTCCAGATGAACTCCCTTAGAGCCGAGGATACTGCT |
| | | | GTCTACTATTGCGCTCGCCAGGGACTCGATTACTGGGGACGGGGCACA |
| | | | CTCGTGACTGTCTCCTCTGTGCGGCCGCCGAGCCCCAAATCTTGTGAC |
| | | | AAAACTCACACATGCCCACCGTGCCCAGCACCTGAACTCCTGGGGGGA |
| | | | CCGTCAGTCTTCCTCTTCCCCCCAAAACCCAAGGACACCCTCATGATCT |
| | | | CCCGGACCCCTGAGGTCACATGCGTGGTGGTGGACGTGAGCCACGAA |
| | | | GACCCTGAGGTCAAGTTCAACTGGTACGTGGACGGCGTGGAGGTGCAT |
| | | | AATGCCAAGACAAAGCCGCGGGAGGAGCAGTACAACAGCACGTACCG |
| | | | TGTGGTCAGCGTCCTCACCGTCCTGCACCAGGACTGGCTGAATGGCAA |
| | | | GGAGTACAAGTGCAAGGTCTCCAACAAAGCCCTCCCAGCCCCCATCGA |
| | | | GAAAACCATCTCCAAAGCCAAAGGGCAGCCCCGAGAACCACAGGTGTA |
| | | | CACCCTGCCCCCATCCCGGGAGGAAATGACCAAAAACCAGGTCAGCCT |
| | | | GACCTGCCTGGTCAAAGGCCTTCTATCCAGCGACATCGCCGTGGAGTG |
| | | | GGAGAGCAATGGGCAGCCGGAGAACAACTACAAGACCACGCCTCCCG |
| | | | TGCTGGACTCCGACGGCTCCTTCTTCCTCTACAGCAAGCTCACCGTGG |
| | | | ACAAGAGCAGGTGGCAGCAGGGGAACGTCTTCTCATGCTCCGTGATGC |
| | | | ATGAGGCTCTGCACAACCACTACACGCAGAAGAGCCTCTCCCTGTCTC |
| | | | CGGGTAAGGATCCCAAACTCTGCTACTTTTGGGTGCTGGTGGTGGTTG |
| | | | GTGGAGTCCTGGCTTGCTATAGCTTGCTAGTAACAGTGGCCTTTATTAT |
| | | | TTTCTGGGTGAGGAGTAAGAGGAGCAGGCTCCTGCACAGTGACTACAT |
| | | | GAACATGACTCCCCGCCGCCCCGGGCCCACCCGCAAGCATTACCAGC |
| | | | CCTATGCCCCACCACGCGACTTCGCAGCCTATCGCTCCAGAGTGAAGT |
| | | | TCAGCAGGAGCGCAGACGCCCCCGCGTACCAGCAGGGCCAGAACCAG |
| | | | CTCTATAACGAGCTCAATCTAGGACGAAGAGAGGAGTACGATGTTTTGG |
| | | | ACAAGAGACGTGGCCGGGACCCTGAGATGGGGGGAAAGCCGCAGAGA |
| | | | AGGAAGAACCCTCAGGAAGGCCTGTACAATGAACTGCAGAAAGATAAG |
| | | | ATGGCGGAGGCCTACAGTGAGATTGGGATGAAAGGCGAGCGCCGGAG |
| | | | GGGCAAGGGGCACGATGGCCTTTACCAGGGTCTCAGTACAGCCACCA |
| | | | AGGACACCTACGACGCCCTTCATATGCAGGCTCTGCCACCTAGOGGAT |
| | | | CCGGCGCAACAAACTTCTCTCTGCTGAAACAAGCCGGAGATGTCGAAG |
| | | | AGAATCCTGGACCGATGCAGATCCCACAGGCGCCCTGGCCAGTCGTCT |
| | | | GGGCGGTGCTACAACTGGGCTGGCGGCCAGGATGGTTCTTAGACTCC |
| | | | CCAGACAGGCCCTGGAACCCCCCCACCTTCTCCCCAGCCCTGCTCGTG |
| | | | GTGACCGAAGGGGACAACGCCACCTTCACCTGCAGCTTCTCCAACACA |
| | | | TCGGAGAGCTTCGTGCTAAACTGGTACCGCATGAGCCCCAGCAACCAG |
| | | | ACGGACAAGCTGGCCGCCTTCCCCGAGGACCGCAGCCAGCCCGGCCA |
| | | | GGACTGCCGCTTCCGTGTCACACAACTGCCCAACGGGCGTGACTTCCA |
| | | | CATGAGCGTTGTCAGGGCCCGGCGCAATGACAGCGGCACCTACCTCT |
| | | | GTGGGGCCATCTCCCTGGCCCCCAAGGCGCAGATCAAAGAGAGCCTG |
| | | | CGGGCAGAGCTCAGGGTGACAGAGAGAAGGGCAGAAGTGCCCACAGC |
| | | | CCACCCCAGCCCCTCACCCAGGCCAGCCGGCCAGTTCCAAACCCTGG |
| | | | TGGTTGGTGTCGTGGGCGGCCTGCTGGGCAGCCTGGTGCTGCTAGTC |
| | | | TGGGTCCTGGCCGTCATCTGCTCCCGGGCCGCACGAGGGTGATAATC |
| | | | CCAGTGTGGTGGTACGGAATTCTCTAGACTGCCGGATCCAAGCTGGAG |
| | | | GAAAAACTGTTTCATACAGAAGGCGTGGAGGAAAAACTGTTTCATACAG |
| | | | AAGGCGTGGAGGAAAAACTGTTTCATACAGAAGGCGTGGAGGAAAAAC |
| | | | TGTTTCATACAGAAGGCGTCGCGAATTCGCGGAGACTCTAGAGGGTAT |
| | | | ATAATGGAAGCTCGATTTCCAGCTTGGCATTCCGGTACTGTTGGTAAAC |
| | | | ACCAAGCTTCACCATGGTGAGCAAGGGCGAGGAGCTGTTCACCGGGG |
| | | | TGGTGATGGCACCAAGAAGGGCTCGCGGATGCCGGACCCTCGGTCTC |
| | | | CCGGCGCTGCTTCTGCTGCTGCTGCTCCGGCCGCCGGCGACGCGGG |
| | | | GCATCACGTGTCCTCCACCCATGTCCGTGGAACACGCAGACATCTGGG |
| | | | TCAAGAGCTACAGCTTGTACTCCAGGGAGCGGTACATTTGTAACTCTGG |
| | | | TTTCAAGAGGAAAGCCGGCACGTCCAGCCTGACGGAGTGCGTGTTGAA |
| | | | CAAGGCCACGAATGTCGCCCACTGGACAACCCCCAGTCTCAAATGCAT |
| | | | TAGAGACCCTGCCCTGGTTCACCAAAGGCCAGCGCCACCCAGCGGCG |
| | | | GAGGATGTGGAGGCGGAGGCTGTGGCGGAAGCGGAGGCGGAGGCAG |
| | | | TATCCAGAACTGGGTGAATGTCATAAGTGATCTGAAGAAAATTGAAGAT |
| | | | CTTATTCAGTCTATGCATATTGATGCTACTCTATATACTGAAAGTGATGT |
| | | | TCACCCCAGTTGCAAAGTGACAGCAATGAAGTGCTTTCTCTTGGAGCTG |
| | | | CAAGTTATTTCACTTGAGTCCGGAGATGCAAGTATTCATGATACAGTGG |
| | | | AAAATCTGATCATCCTTGCAAACGACAGTTTGTCTTCTAATGGGAATGTA |
| | | | ACAGAATCTGGATGCAAAGAATGTGAGGAACTGGAGGAAAAAAATATTA |
| | | | AAGAATTTTTGCAGAGTTTTGTACATATTGTCCAAATGTTCATCAACACT |
| | | | TCTTGATAA |

Preferred Amino Acid Sequences of the Invention:

| SEQ | | | |
|---|---|---|---|
| ID | Info | Specificity | Amino Acid Sequence |
| 14 | First protein | CAR | MGWSCIILFLVATTATGVHS |
| | signal | | |
| | sequence | | |
| 15 | VL variable | CD44v6 | EIVLTQSPATLSLSPGERATLSCSASSSINYIYWYQQKPGQAPRLLIYLTSNL |
| | domain | | ASGVPARFSGSGSGTDFTLTISSLEPEDFAVYYCLQWSSNPLTFGGGTKV |
| | | | EIKGSTSGSGKPGSGEGSTKGQVQLQ |
| 16 | VL variable | CDR-1 | SSINYIY |
| | domain | | |
| 17 | VL variable | CDR-2 | LLIYLTSNLAS |
| | domain | | |
| 18 | VL variable | CDR-3 | LQWSSNPL |
| | domain | | |
| 19 | VH variable | CD44v6 | EVQLVESGGGLVKPGGSLRLSCAASGFTFSSYDMSWVRQAPGKGLEWVS |
| | domain | | TISSGGSYTYYLDSIKGRFTISRDNAKNSLYLQMNSLRAEDTAVYYCARQG |
| | | | LDYWGRGTLVTVSSVRPPS |
| 20 | VH variable | CDR-1 | FTFSSYDMS |
| | domain | | |
| 21 | VH variable | CDR-2 | WVSTISSGGSYTYYL |
| | domain | | |
| 22 | VH variable | CDR-3 | RQGLDY |
| | domain | | |
| 23 | Immunoglobul | | PKSCDKTHTCPPCPAPELLGGPSVFLFPPKPKDTLMISRTPEVTCVVVDVS |
| | in heavy chain | | HEDPEVKFNWYVDGVEVHNAKTKPREEQYNSTYRVVSVLTVLHQDWLNG |
| | extracellular | | KEYKCKVSNKALPAPIEKTISKAKGQPREPQVYTLPPSREEMTKNQVSLTC |
| | constant | | LVKGLLSSDIAVEWESNGQPENNYKTTPPVLDSDGSFFLYSKLTVDKSRW |
| | region (CAR) | | QQGNVFSCSVMHEALHNHYTQKSLSLSPGK |
| 24 | T cell Co- | CD28 | DPKLCYFWVLVVVGGVLACYSLLVTVAFIIFWVRSKRSRLLHSDYMNMTPR |
| | stimulatory | | RPGPTRKHYQPYAPPRDFAAYRS |
| | signaling | | |
| | domain (CAR) | | |
| 25 | Transmembra | CD28 | WVLVVVGGVLACYSLLVTVAFII |
| | ne sequence; | | |
| | from within | | |
| | SEQ ID NO | | |
| | 24 | | |
| 26 | T cell | CD3 zeta | RVKFSRSADAPAYQQGQNQLYNELNLGRREEYDVLDKRRGRDPEMGGK |
| | activation | signaling | PQRRKNPQEGLYNELQKDKMAEAYSEIGMKGERRRGKGHDGLYQGLSTA |
| | signaling | domain | TKDTYDALHMQALPPS |
| | domain (CAR) | (CAR) | |
| 27 | Recombinant | P2A | GSGATNFSLLKQAGDVEENPGP |
| | protein | cleavage | |
| | cleavage motif | sequence | |
| 28 | Dominant | Dominant | MQIPQAPWPVVWAVLQLGWRPGWFLDSPDRPWNPPTFSPALLVVTEGD |
| | negative | negative | NATFTCSFSNTSESFVLNWYRMSPSNQTDKLAAFPEDRSQPGQDCRFRV |
| | truncated | truncated | TQLPNGRDFHMSVVRARRNDSGTYLCGAISLAPKAQIKESLRAELRVTERR |
| | Checkpoint | PD1 | AEVPTAHPSPSPRPAGQFQTLVVGVVGGLLGSLVLLVWVLAVICSRAARG |
| | inhibitor | | |
| 29 | Second | IL-15 | MVSKGEELFTGVVMAPRRARGCRTLGLPALLLLLLLRPPATR |
| | protein Signal | | |
| | sequence | | |
| | (Cytokine) | | |
| 30 | Cytokine RA | IL15RA N- | GITCPPPMSVEHADIWVKSYSLYSRERYICNSGFKRKAGTSSLTECVLNKA |
| | N-terminal | terminal 120 | TNVAHWTTPSLKCIRDPALVHQRPAPP |
| | | amino acids | |
| 31 | Linker | Linking loop | SGGGGGGGGCGGSGGGGS |
| | Sequence | sequence | |
| 32 | Cytokine | IL-15 | IQNWVNVISDLKKIEDLIQSMHIDATLYTESDVHPSCKVTAMKCFLLELQVIS |
| | | | LESGDASIHDTVENLIILANDSLSSNGNVTESGCKECEELEEKNIKEFLQSFV |
| | | | HIVQMFINTS |
| 33 | CAR | CD44v6 | MGWSCIILFLVATTATGVHSEIVLTQSPATLSLSPGERATLSCSASSSINYIY |
| | | | WYQQKPGQAPRLLIYLTSNLASGVPARFSGSGSGTDFTLTISSLEPEDFAV |
| | | | YYCLQWSSNPLTFGGGTKVEIKGSTSGSGKPGSGEGSTKGQVQLQEVQL |
| | | | VESGGGLVKPGGSLRLSCAASGFTFSSYDMSWVRQAPGKGLEWVSTISS |
| | | | GGSYTYYLDSIKGRFTISRDNAKNSLYLQMNSLRAEDTAVYYCARQGLDY |
| | | | WGRGTLVTVSSVRPPSPKSCDKTHTCPPCPAPELLGGPSVFLFPPKPKDT |
| | | | LMISRTPEVTCVVVDVSHEDPEVKFNWYVDGVEVHNAKTKPREEQYNSTY |
| | | | RVVSVLTVLHQDWLNGKEYKCKVSNKALPAPIEKTISKAKGQPREPQVYTL |
| | | | PPSREEMTKNQVSLTCLVKGLLSSDIAVEWESNGQPENNYKTTPPVLDSD |
| | | | GSFFLYSKLTVDKSRWQQGNVFSCSVMHEALHNHYTQKSLSLSPGKDPKL |
| | | | CYFWVLVVVGGVLACYSLLVTVAFIIFWVRSKRSRLLHSDYMNMTPRRPG |
| | | | PTRKHYQPYAPPRDFAAYRSRVKFSRSADAPAYQQGQNQLYNELNLGRR |
| | | | EEYDVLDKRRGRDPEMGGKPQRRKNPQEGLYNELQKDKMAEAYSEIGMK |
| | | | GERRRGKGHDGLYQGLSTATKDTYDALHMQALPPS |
| 34 | Dominant | Dominant | MQIPQAPWPVVWAVLQLGWRPGWFLDSPDRPWNPPTFSPALLVVTEGD |
| | negative | negative | NATFTCSFSNTSESFVLNWYRMSPSNQTDKLAAFPEDRSQPGQDCRFRV |
| | truncated | truncated | TQLPNGRDFHMSVVRARRNDSGTYLCGAISLAPKAQIKESLRAELRVTERR |
| | Checkpoint | PD1 | AEVPTAHPSPSPRPAGQFQTLVVGVVGGLLGSLVLLVWVLAVICSRAARG |
| | inhibitor | | |
| 35 | Cytokine | IL-15 fusion | MVSKGEELFTGVVMAPRRARGCRTLGLPALLLLLLLRPPATRGITCPPPMS |
| | | protein | VEHADIWVKSYSLYSRERYICNSGFKRKAGTSSLTECVLNKATNVAHWTTP |
| | | | SLKCIRDPALVHQRPAPPSGGGGGGGGCGGSGGGGSIQNWVNVISDLKKI |
| | | | EDLIQSMHIDATLYTESDVHPSCKVTAMKCFLLELQVISLESGDASIHDTVE |
| | | | NLIILANDSLSSNGNVTESGCKECEELEEKNIKEFLQSFVHIVQMFINTS |
| 36 | VL variable | CEA | DIQMTQSPSSLSASVGDRVTITCSTSSSVSYMHWYQQKPGKAPRLLIYSTS |
| | domain | | NLASGVPSRFSGSGSGTDFTFTISSLQPEDIATYYCHQWSSYPTFGQGTK |
| | | | VEIKGSTSGSGKPGSGEGSTKGQVQLQ |
| 37 | VL variable | CDR-1 | SSVSYMH |
| | domain | | |
| 38 | VL variable | CDR-2 | LLIYSTSNLAS |
| | domain | | |
| 39 | VL variable | CDR-3 | HQWSSYP |
| | domain | | |
| 40 | VH variable | CEA Was | ESGPGLVRPSQTLSLTCTVSGFTISSGYSWHWVRQPPGRGLEWIGYIQYS |
| | domain | soll das | GITNYNPSLKSRVTMLVDTSKNQFSLRLSSVTAADTAVYYCAREDYDYHW |
| | | hier? | YFDVWGQGSTVTVSSGAAAE |
| 41 | VH variable | CDR-1 | FTISSGYSWH |
| | domain | | |
| 42 | VH variable | CDR-2 | WIGYIQYSGITNY |
| | domain | | |
| 43 | VH variable | CDR-3 | REDYDYHWYFDV |
| | domain | | |
| 44 | CAR variable | CEA | GACATCCAGATGACCCAGAGCCCAAGCAGCCTGAGCGCCAGCGTGGG |
| | domain | | TGACAGAGTGACCATCACCTGTAGTACCAGCTCGAGTGTAAGTTACATG |
| | | | CACTGGTACCAGCAGAAGCCAGGTAAGGCTCCAAGGCTGCTGATCTAC |
| | | | AGCACATCCAACCTGGCTTCTGGTGTGCCAAGCAGATTCAGCGGTAGC |
| | | | GGTAGCGGTACCGACTTCACCTTCACCATCAGCAGCCTCCAGCCAGAG |
| | | | GACATCGCCACCTACTACTGCCATCAGTGGAGTAGTTATCCCACGTTCG |
| | | | GCCAAGGGACCAAGGTGGAAATCAAAGGATCCACTTCCGGTTCAGGAA |
| | | | AGCCCGGGAGTGGTGAAGGTAGCACTAAAGGCCAGGTCCAGCTGCAG |
| | | | GAGAGCGGTCCAGGTCTTGTGAGACCTAGCCAGACCCTGAGCCTGAC |
| | | | CTGCACCGTGTCTGGCTTCACCATCAGCAGTGGTTATAGCTGGCACTG |
| | | | GGTGAGACAGCCACCTGGACGAGGTCTTGAGTGGATTGGATACATACA |
| | | | GTACAGTGGTATCACTAACTACAACCCCTCTCTCAAAAGTAGAGTGACA |
| | | | ATGCTGGTAGACACCAGCAAGAACCAGTTCAGCCTGAGACTCAGCAGC |
| | | | GTGACAGCCGCCGACACCGCGGTCTATTATTGTGCAAGAGAAGACTAT |
| | | | GATTACCACTGGTACTTCGATGTCTGGGGTCAAGGCAGCACGGTCACC |
| | | | GTCTCCTCAGGTGCGGCCGCCGAG |

### FIGURES

The invention is demonstrated by way of the example through the figures disclosed herein. The figures provided represent particular, non-limiting embodiments and are not intended to limit the scope of the invention.

### SHORT DESCRIPTION OF THE FIGURES

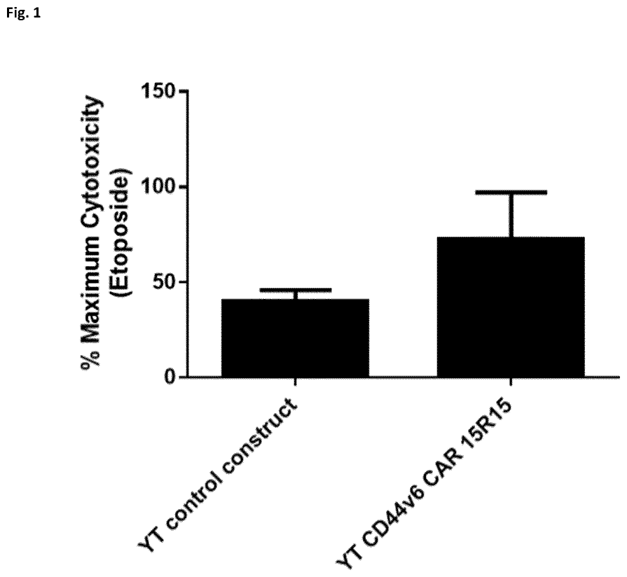

FIG. 1: Cytotoxicity of CD44v6 CAR-transduced YT cells to MCF-7 cells.

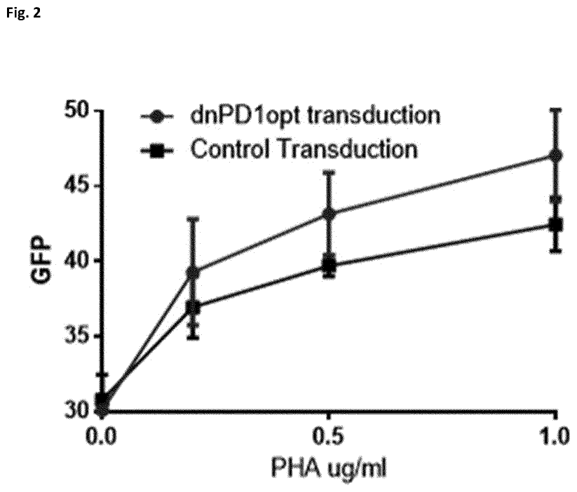

FIG. 2: Checkpoint inhibition by a dominant negative PD1 (dnPD1opt) leads to an improvement in NFAT promoter activity.

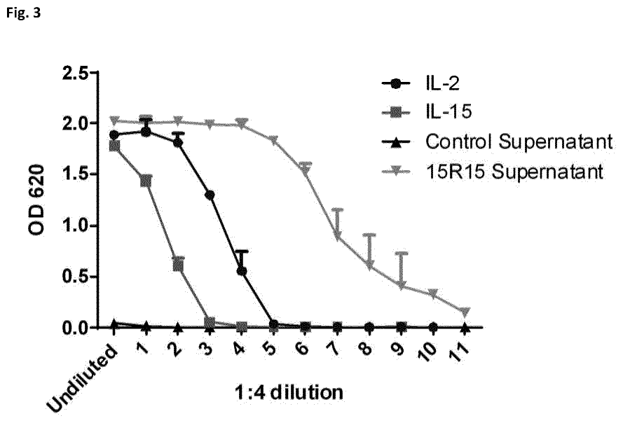

FIG. 3: The activity IL-15 superagonists (15R15) compared to IL-2 or IL-I5.

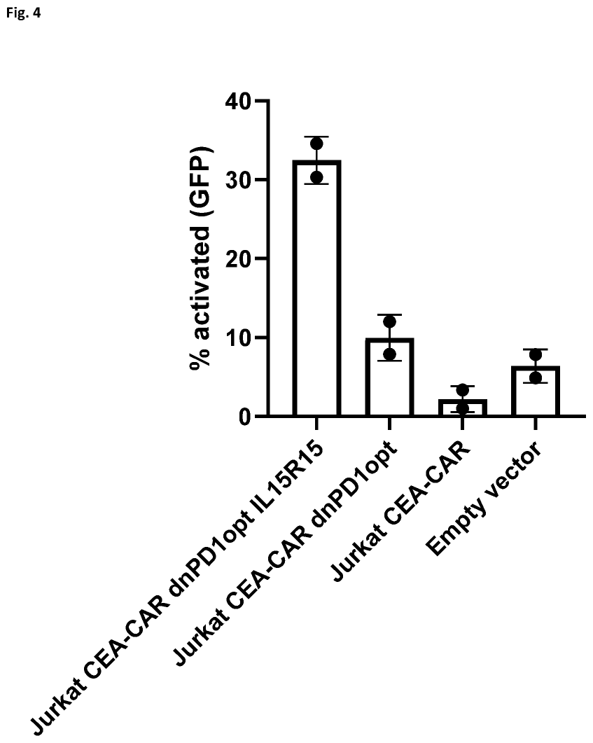

FIG. 4: The NF-kB promoter activity of anti-CEA CAR expressing Jurkat cells stimulates with target cells (MCF-7) with or without checkpoint inhibition (dnPD1opt) or IL-IS superagonists (15R15).

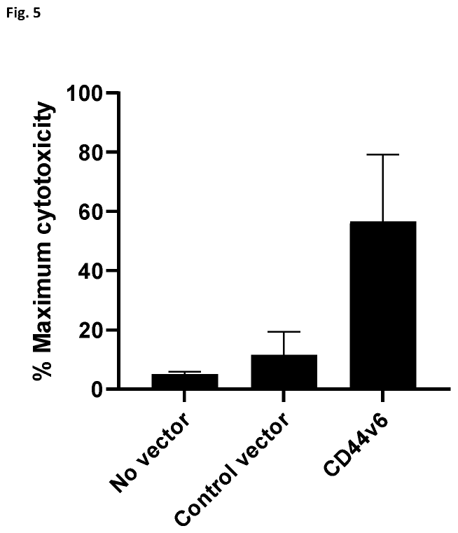

FIG. 5: Specific kill of MCF-7 cells with primary NK cells transduced with CD44v6 CAR construct.

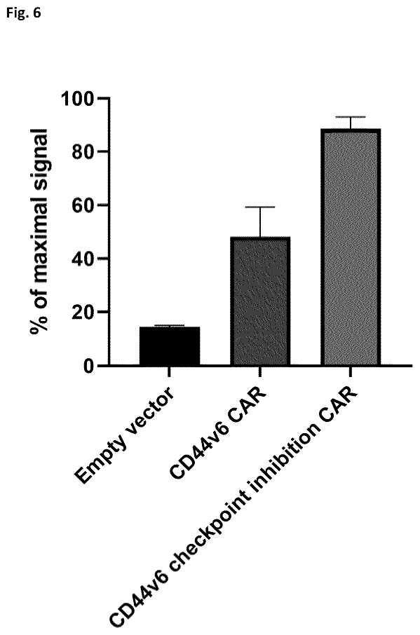

FIG. 6: PD1 transfected reporter Jurkat cells stimulated with MCF-7.

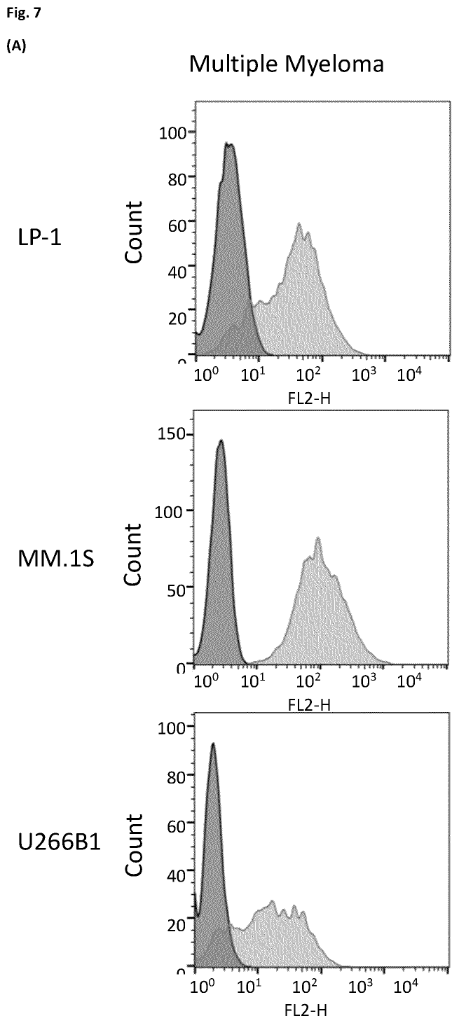

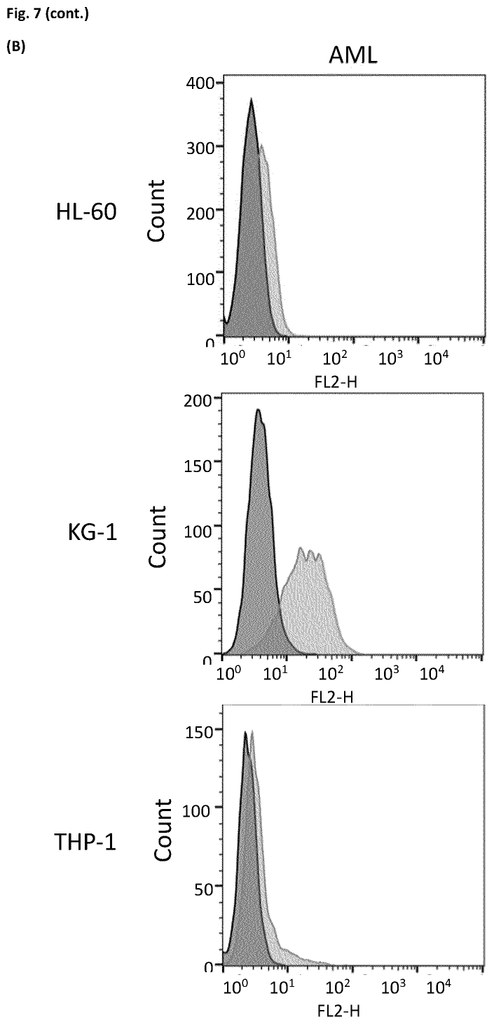

FIG. 7: Evaluation of disease indication by CD44v6 expression analysis.

### DETAILED DESCRIPTION OF THE FIGURES

FIG. 1: Cytotoxicity of CD44v6 CAR-transduced YT cells to MCF-7 cells: YT cells were transduced with CD44v6 CAR construct encoding lentivirus with dnPD1opt and 15R15 (YT CD44v6 CAR 15R15) or empty vector (YT control construct). YT CD44v6 CAR 15R15 cells or YT control construct were then added to duplicate wells of a 96-well plate containing MCF-7 cells, and a non-specific cytotoxic signal (etoposide 10 ug/ml) was added to further wells. Cytotoxicity was determined after 18 hours.

FIG. 2: Checkpoint inhibition by a dominant negative PD1 (dnPD1opt) leads to an improvement in NFAT promoter activity: Jurkat cells expressing GFP under the control of the NFAT promoter were transduced with a dentPD1opt expressing lentiviral vector or a control vector. The cells were then exposed 1 day earlier to a cell line expressing high levels of PD-L1 (U251-PD-L1) in duplicate wells of a 96-well plate with increasing levels of a TCR-linked proliferative signal (phytohemagglutinin: PHA). The number of GFP expressing cells was determined by flow cytometry.

FIG. 3: The activity IL-I5 superagonists (15R15) compared to IL-2 or IL-I5: The IL-15 transgene 15R15 or an empty plasmid was expressed in HEK293 cells after transient transfection and collected 2 days after transfection. The supernatant was double tested for IL-2/IL-15 specific activity in a bioassay using the Hek-Blue IL-2 reporter cell line as described by the manufacturer (Invivogen).

FIG. 4: The NF-kB promoter activity of anti-CEA CAR expressing Jurkat cells stimulates with target cells (MCF-7) with or without checkpoint inhibition (dnPD1opt) or IL-15 superagonists (15R15): Jurkat cells expressing GFP under the control of an NF-kB promoter were transduced with a lentiviral vector encoding (1) a CEA-CAR construct combined with dnPD1opt and 15R15 (Jurkat-CEA-CAR-dnPD1opt-IL15R15), (2) a CEA-CAR construct combined with dnPD1opt (Jurkat-CEA-CAR-dnPD1opt), (3) a CEA-CAR construct (Jurkat CEA-CAR) or (4) an empty lentiviral vector (empty vector). The transduced Jurkat cells were transferred to MCF-7 target cells. After 1 day, positive cells were analysed by flow cytometry.

FIG. 5: Specific kill of MCF-7 cells with primary NK cells transduced with CD44v6 CAR construct: Primary NK cells were transduced with CD44v6 CAR construct or empty vector and compared with non-transduced primary NK cells. Cells were added to duplicate wells of a 96-well plate containing MCF-7 cells, and a non-specific cytotoxic signal (etoposide 10 ug/ml) was added to further wells. Cytotoxicity was determined after 18 hours.

FIG. 6: PD1 transfected reporter Jurkat cells stimulated with MCF-7: PD1 transfected reported Jurkat cells were transduced with a lentiviral vector under the control of an SSFV promoter encoding (1) an empty lentiviral vector (empty vector, left bar), (2) a CD44v6-CAR (CD44v6 CAR, middle bar) or (3) a CD44v6-CAR construct combined with dnPD1opt (CD44v6 checkpoint inhibition CAR, right bar). The transduced Jurkat cells were transferred to MCF-7 target cells. Signal intensity of positive cells was analysed by flow cytometry.

FIG. 7: Evaluation of disease indication by CD44v6 expression analysis: CD44v6 is highly expressed in different (A) Multiple Myeloma cell lines and (B) AML cell lines.

### EXAMPLES

The invention is demonstrated through the examples disclosed herein. The examples provided represent particular embodiments and are not intended to limit the scope of the invention. The examples are to be considered as providing a non-limiting illustration and technical support for carrying out the invention.

Lentiviral vectors encoding the anti-CD44v6 CAR, a checkpoint inhibitor, the dominant negative truncated PD-1 protein, an immunostimulatory cytokine, the IL-15 transgenic 15R15, or a combination of these, are introduced into immune cell lines using the well-known method of lentiviral gene transfer. The cell line modified in this way lyses the CD44v6-positive cancer cell line MCF-7, while the immune cell line YT Is not modified with the anti-CD44v6 CAR and cannot sufficiently lyse the cancer cell line MCF-7 (Example 1, FIG. 1).

Further examples show
the successful PD-1 checkpoint inhibition (Example 2, FIG. 2),
the high activity of the IL-15 superagonist (Example 3, FIG. 3),
the killing activity in primary NK cells (Example 1, FIG. 5),
the CD44v6 CAR expression in liquid cancer cell lines (Example 5, FIG. 7)

According to Dull et al. (1998), the packaging, production and cell transduction of lentiviruses was carried out using the described high-security 3rd generation plasmid system. The cells transfection was performed using polyethyleneimine according to the manufacturer's instructions (Polyplus).

Hek 293T and MC32A cells were cultivated in DMEM with 10% heat-inactivated FCS and penicillin/streptomycin. Jurkat cells were cultured in RPMI with 10% heat-inactivated FCS and penicillin/streptomycin. YT cells were cultivated in RPMI with 10% heat-inactivated FCS, penicillin/streptomycin and 10 IU/ml IL-2. GFP was measured by flow cytometry using a FACS-Calibur. Cytotoxicity was determined by a crystal violet assay or a chromium release assay according to standard procedures.

### Example 1: Cytotoxicity of YT Cells to MCF-7 Cells Wherein YT Cells are Transduced with Anti-CD44v6 CAR Construct

This example refers to experimental results with YT cells transduced with empty a lenitviral vector (YT control construct) or a lentiviral vector comprising nucleic acid sequence regions encoding a chimeric antigen receptor (CAR) binding to CD44v6, a checkpoint inhibitor dnPD1opt, and/or an immune stimulatory cytokine 15R15 (YT CD44v6 CAR 15R15). YT CD44v6 CAR 15R15 cells or YT control construct cells were then added in duplicates to a 96-well plate containing MCF-7 cells, and a non-specific cytotoxic signal (etoposide 10 ug/ml) was added to further wells. Cytotoxicity was determined after 18 hours. The DNA sequence of the CAR was transferred to the immune cell line YT using the well-known method of lentiviral gene transfer. The cell line modified in this way lyses the CD44v6-positive cancer cell line MCF-7. The immune cell line YT, which is not modified with the CAR, does not sufficiently lyse the cancer cell line MCF-7 (FIG. 1).

Primary NK cells transduced with CD44v6 CAR construct efficiently lyses the CD44v6-positive cancer cell line MCF-7 compared to cells transduced with an empty vector or with non-transduced primary NK cells (FIG. 5).

### Example 2: Checkpoint Inhibition by a Dominant Negative PD1 (dnPD1opt) Leads to an Improvement in NFAT Promoter Activity

The example relates to experiments that show successful inhibition of the checkpoint protein PD-1 through the dominant negative truncated form of PD-1, dntPD1opt. Jurkat cells expressing GFP under the control of the NFAT promoter (FIG. 2). The Jurkat cells were transduced with a control lentiviral vector or the checkpoint inhibitor (dntPD1opt) encoding lentiviral vector. One day before, these cells were exposed in duplicate in a 96 well plate to a cell line expressing high concentrations of PD-L1 (U251-PD-L1) and an increasing concentration of a TCR mediated T cell stimulus, phytohaemagglutinin (PHA). The number of GFP expressing cells, determined by flow cytometry, was higher for dntPD1opt expressing Jurkat cells compared to the negative control. This demonstrates a successful inhibition of the checkpoint protein PD-1 at biological relevant levels. Example 3: The activity IL-15 superagonists (15R15) compared to IL-2 or IL-15

The example relates to experiments that proves superagonist activity of the IL-15 transgene 15R15 (FIG. 3). The IL-15 transgene 15R15 or an empty plasmid was expressed in HEK293 cells after transient transfection and collected 2 days after transfection. The supernatant was tested twice for IL-2/IL-15 specific activity in a bioassay using the Hek-Blue IL-2 reporter cell line as described by the manufacturer (Invivogen). The OD260 was measured. This demonstrates successfully a superagonist activity of the IL-15 transgene 15R15 that is higher than the negative control, IL-2, and IL-15 at biological relevant levels.

### Example 4: The NF-kB Promoter Activity of Anti-CEA CAR Expressing Jurkat Cells Stimulates with Target Cells (MCF-7) with or without Checkpoint Inhibition (dnPD1opt) or IL-15 Superagonists (15R15)

In a further example, the inventors employed an antigen binding domain in the CAR that binds CEA. The carcinoembryonic antigen (CEA) is a transmembrane glycoprotein of the immunoglobulin superfamily. CEA is found both as a soluble form in blood and bound to cell membranes, mostly tumor cells. As a cell surface glycoprotein, the protein is involved in cell adhesion, intracellular signal transduction and tumor progression. CEA in its soluble form is used as a clinical biomarker for solid, especially malignant tumors, such as gastrointestinal cancers and may promote tumor development through its role as a cell adhesion molecule. It also plays a role as an oncogene by promoting tumor progression and inducing resistance of colorectal cancer cells to therapy. In addition, the encoded protein can regulate differentiation, apoptosis and cell polarity. Currently, no curative therapy exists for CEA high-positive tumor stem cells.

The remaining components of the CAR constructs were the same as those employed in the other examples. The antigen-binding domain of the CAR was adjusted to bind CEA. The employed CAR sequences are disclosed in the table above under SEQ ID NO 36-43, the coding sequence for the complete variable domain coding the antigen binding domain is disclosed in SEQ ID NO 44.

The example relates therefore to experiments showing a successful NF-kB promoter activity in Jurkat cells expressing anti-CEA CAR, checkpoint inhibitor dnPD1opt, and IL-15 transgene 15R15 and challenged with MCF-7 target cells. Refer FIG. 4.

GFP expression under the control of the NF-kB promoter was determined in Jurkat cells transduced with a lentiviral vector encoding (1) an anti-CEA CAR construct combined with checkpoint inhibitor dntPD1opt and IL-15 transgene 15R15 (Jurkat-CEA-CAR-dnPD1opt-IL15R15) or (2) an anti-CEA CAR construct combined with checkpoint inhibitor dntPD1opt (Jurkat-CEA-CAR-dnPD1opt), (3) an anti-CEA-CAR (Jurkat CEA-CAR) or (4) an empty lentiviral vector (empty vector). The transduced Jurkat cells were transferred to MCF-7 target cells. After 1 day, GFP positive cells were determined by flow cytometry.

The combination Jurkat-CEA-CAR-dnPD1opt-IL15R15 shows GFP expression at a very high level and higher than in Jurkat-CEA-CAR-dnPD1opt, Jurkat CEA-CAR, empty vector samples. The GFP expression level in Jurkat-CEA-CAR-dnPD1opt-IL15R15 is greater than an additive effect and thus proves a synergistic effect of the combination described in this invention.

The example also relates therefore to another experiment showing a successful SFFV promoter activity in Jurkat cells expressing CD44v6 CAR or CD44v6 checkpoint inhibition CAR and challenged with MCF-7 target cells. Refer FIG. 6.

### Example 5: Evaluation of Disease Indication by CD44v6 Expression Analysis

The CAR target CD44v6 is strongly expressed on MM, Lymphomas, and Leukemias (FIG. 7), but is absent or weakly expressed in normal cells (non-cancerous cells). CD44v6 drives growth of cancer-initiating stem cells and tumor progression. Therefore, the CD44v6 CAR presents a high safety profile. For example, CD44v6-targeted T cells do not recognize hematopoietic stem cells and keratinocytes and can cause reversible monocytopenia.

### REFERENCES

Kreye, J., et al Human cerebrospinal fluid monoclonal N-methyl-D-aspartate receptor autoantibodies are sufficient for encephalitis pathogenesis. Brain 139, 2641-2652 (2016).

1. A recombinant nucleic acid expression construct, comprising:
(a.) a first nucleic acid sequence region encoding a chimeric antigen receptor (CAR),
(b.) a second nucleic acid sequence region encoding a checkpoint inhibitory molecule, and
(c.) a third nucleic acid sequence region encoding an immune stimulatory cytokine.

2. The recombinant nucleic acid expression construct according to claim 1, wherein the first nucleic acid sequence region encoding the CAR comprises:
(d.) a nucleic acid sequence encoding an extracellular antigen-binding domain, said antigen-binding domain preferably comprising an antibody or antibody fragment,
(e.) a nucleic acid sequence encoding a transmembrane domain, and
(f.) a nucleic acid sequence encoding an intracellular co-stimulatory domain.

3. The recombinant nucleic acid expression construct according to claim 1, wherein at least the first nucleic acid sequence region encoding the CAR is constitutively expressed by a promoter or promoter/enhancer combination.

4. (canceled)

5. The recombinant nucleic acid expression construct according to claim 1, wherein at least the first nucleic acid sequence region encoding the CAR and the second nucleic acid sequence region encoding the checkpoint inhibitory molecule, are configured to encode a polycistronic mRNA comprising coding regions for the polypeptide sequences of the CAR and the checkpoint inhibitory molecule, and wherein an amino acid sequence comprising a polypeptide cleavage site is disposed between the CAR polypeptide and the checkpoint inhibitory molecule polypeptide.

6. The recombinant nucleic acid expression construct according to claim 5, wherein the polypeptide cleavage site is selected from the group consisting of P2A, T2A, E2A and F2A.

7. The recombinant nucleic acid expression construct according to claim 1, wherein the checkpoint inhibitory molecule encoded by the second nucleic acid sequence region is a dominant negative polypeptide and/or an antibody inhibiting and/or blocking an immune checkpoint protein.

8. The recombinant nucleic acid expression construct according to claim 7, wherein the checkpoint inhibitory polypeptide is a dominant negative truncated PD1 polypeptide or a PD1 antibody.

9. The recombinant nucleic acid expression construct according to claim 1, wherein the third nucleic acid sequence region encoding an immune stimulatory cytokine comprises a nucleic acid sequence encoding one or more immune stimulatory cytokines operably linked to one or more promoters, wherein at least one of said cytokines is selected from the group consisting of IL-15, IL-15RA, IL-2, IL-7, IL-12, IL-21, IFN gamma, and IFN beta.

10. The recombinant nucleic acid expression construct according to claim 9, wherein the third nucleic acid sequence region encoding the immune stimulatory cytokine is operably linked to one or more constitutive promoters, and wherein the immune stimulatory cytokine maintains or enhances the activity, survival and/or number of immune cells within and/or in proximity to tumor tissue.

11. The recombinant nucleic acid expression construct according to claim 1, wherein said construct optionally comprises an additional nucleic acid sequence region encoding a chemokine receptor.

12. The recombinant nucleic acid expression construct according to claim 11, wherein the chemokine receptor is C—C chemokine receptor type 4 (CCR4).

13. The recombinant nucleic acid expression construct according to claim 1, wherein said construct optionally comprises a further nucleic acid sequence region encoding a suicide gene.

14. The recombinant nucleic acid expression construct according to claim 1, wherein the extracellular antigen-binding domain of the CAR encoded by the first nucleic acid sequence region specifically recognizes human CD44v6.

15. The recombinant nucleic acid expression construct according to claim 14, wherein the said CAR comprises:
a CAR signal sequence;
an antigen-binding domain of a CAR that specifically recognizes CD44v6;
an immunoglobulin heavy chain extracellular constant region of a CAR;
a CD28 signalling domain; wherein the CD28 signaling domain comprises a transmembrane domain; and
a CD3 zeta signaling domain.

16. The recombinant nucleic acid expression construct according to claim 15, wherein the CAR comprises:
a CAR signal sequence according to SEQ ID NO 14 or a sequence with at least 80% sequence identity to SEQ ID NO 14;
an antigen-binding domain of a CAR that specifically recognizes CD44v6 according to SEQ ID NO 15 and SEQ ID NO 19 or a sequence with at least 80% sequence identity to SEQ ID NO 15 and 19;
an immunoglobulin heavy chain extracellular constant region of a CAR according to SEQ ID NO 23 or a sequence with at least 80% sequence identity to SEQ ID NO 23;
a CD28 signalling domain according to SEQ ID NO 24 or a sequence with at least 80% sequence identity to SEQ ID NO 24; wherein the CD28 signaling domain comprises a transmembrane domain according to SEQ ID NO 25 or a sequence with at least 80% sequence identity to SEQ ID NO 25; and
a CD3 zeta signaling domain according to SEQ ID NO 26 or a sequence with at least 80% sequence identity to SEQ ID NO 26.

17. (canceled)

18. The recombinant nucleic acid expression construct according to claim 1, wherein the checkpoint inhibitory molecule comprises:
a dominant negative truncated form of a checkpoint protein,
wherein said checkpoint protein is positioned adjacently to a polypeptide cleavage site for cleaving the checkpoint inhibitory molecule from the CAR polypeptide.

19. The recombinant nucleic acid expression construct according to claim 18, wherein the dominant negative truncated form of a checkpoint protein is dominant negative truncated PD1 according to SEQ ID NO 28 or a sequence with at least 80% sequence identity to SEQ ID NO 28 and wherein the cleavage site is selected from the group consisting of P2A, T2A, E2A and F2A.

20. The recombinant nucleic acid expression construct according to claim 1, wherein the immune stimulatory cytokine comprises:
A signal sequence;
A N-terminal IL15RA polypeptide;
A linking loop sequence; and
An IL-15 polypeptide.

21. The recombinant nucleic acid expression construct according to claim 20, wherein the immune stimulatory cytokine comprises:
A signal sequence according to SEQ ID NO 29 or a sequence with at least 80% sequence identity to SEQ ID NO 29;
A N-terminal IL15RA polypeptide according to SEQ ID NO 30 or a sequence with at least 80% sequence identity to SEQ ID NO 30;
A linking loop sequence according to SEQ ID NO 31 or a sequence with at least 80% sequence identity to SEQ ID NO 31; and
An IL-15 polypeptide according to SEQ ID NO 32 or a sequence with at least 80% sequence identity to SEQ ID NO 32.

22. The recombinant nucleic acid expression construct according to claim 1, wherein said construct comprises nucleic acid sequence regions encoding:
a CAR that specifically recognizes human CD44v6,
a checkpoint inhibitory molecule dominant negative truncated PD1 polypeptide, and
an immune stimulatory cytokine, comprising a signal sequence, a N-terminal IL15RA polypeptide, a linking loop sequence, and an IL-15 polypeptide.

23. The recombinant nucleic acid expression construct according to claim 22, wherein the checkpoint inhibitory molecule is positioned adjacent to a polypeptide cleavage site P2A for cleaving the checkpoint inhibitory molecule from the CAR polypeptide.

24. A chimeric antigen receptor (CAR) polypeptide encoded by the recombinant nucleic acid expression construct according to claim 1.

25. Genetically modified cells, comprising a recombinant nucleic acid expression construct according to claim 1.

26. The genetically modified cells according to claim 25, wherein the cells are selected from the group consisting of induced pluripotent stem cells (iPSC), immortalized immune cells, primary immune cells, Natural Killer (NK) cells, NK T cells, cytokine-induced killer cells (CIK), and T lymphocytes.

27. (canceled)

28. (canceled)

29. The genetically modified cells according to claim 25, wherein the cells are induced pluripotent stem cells (iPSC) line ND50039.

30. (canceled)

31. A method for the treatment of a medical disorder associated with the presence of pathogenic cells expressing CD44v6, comprising administering the genetically modified stem cells according to claim 25 to a subject, wherein the extracellular antigen-binding domain of the CAR specifically recognizes human CD44v6.

32. The method according to claim 31, wherein the medical disorder comprises cancer cells expressing CD44v6.

33. (canceled)

34. (canceled)

35. (canceled)

36. (canceled)

37. A method for producing genetically modified cells , comprising delivering or transferring a nucleic acid construct according to claim 1 into cells in vitro.

38. (canceled)

39. (canceled)

40. A pharmaceutical composition comprising genetically modified cells according to claim 25 and a pharmaceutically acceptable carrier.

41. (canceled)

42. (canceled)

43. (canceled)

44. The method according to claim 37, wherein the nucleic acid construct is transferred or delivered into the cell in vitro using electroporation.


SEQUENCE LISTING (44 sequences)

SEQ ID NO: 1 - DNA, 60 bp, Artificial Sequence
  CAR component
atgggatggagctgtatcatcctcttcctggtagcaacaacagccaccggcgtccatagc

SEQ ID NO: 2 - DNA, 744 bp, Artificial Sequence
  CAR variable
gagatcgtcctgactcagagccctgcaaccctgtccctgtctccaggcgaacgggctacactgagctgctctgcctctagcagcattaactacatttactggtatcagcagaagccaggccaagccccgaggctccttatttacttgacttccaacctggctagtggagtgccggctaggttcagcgggagcggctcaggcacagacttcacattgacaatatcaagcctggaacccgaagattttgctgtgtactattgtctccaatggtcttctaatcctctcacgttcggaggggggactaaggtggagattaaggggtcaacctctggcagcgggaaacctggctctggcgagggcagcaccaagggtcaagtccagctccaagaagtgcaactcgtggaatcaggaggcggcttggtgaagcccggcggctccttgagactgtcttgcgctgctagcgggtttacattctcatcttacgacatgagttgggttcgccaggcacctggaaaaggcctcgagtgggtcagcactatttctagcggcggatcttacacctactatctggacagcatcaaaggacgcttcacaatttctcgcgacaatgctaagaactctttgtatctccagatgaactcccttagagccgaggatactgctgtctactattgcgctcgccagggactcgattactggggacggggcacactcgtgactgtctcctctgtgcggccgccgagc

SEQ ID NO: 3 - DNA, 693 bp, Artificial Sequence
  CAR component
cccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggaaatgaccaaaaaccaggtcagcctgacctgcctggtcaaaggccttctatccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaag

SEQ ID NO: 4 - DNA, 222 bp, Artificial Sequence
  CAR component
gatcccaaactctgctacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 5 - DNA, 339 bp, Artificial Sequence
  CAR component
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcatatgcaggctctgccacctagc

SEQ ID NO: 6 - DNA, 66 bp, Artificial Sequence
  CAR component
ggatccggcgcaacaaacttctctctgctgaaacaagccggagatgtcgaagagaatcctggaccg

SEQ ID NO: 7 - DNA, 597 bp, Artificial Sequence
  CAR construct component
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgttgtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggtga

SEQ ID NO: 8 - DNA, 260 bp, Artificial Sequence
  Promoter
taatcccagtgtggtggtacggaattctctagactgccggatccaagctggaggaaaaactgtttcatacagaaggcgtggaggaaaaactgtttcatacagaaggcgtggaggaaaaactgtttcatacagaaggcgtggaggaaaaactgtttcatacagaaggcgtcgcgaattcgcggagactctagagggtatataatggaagctcgatttccagcttggcattccggtactgttggtaaacaccaagcttcacc

SEQ ID NO: 9 - DNA, 129 bp, Artificial Sequence
  CAR construct component
atggtgagcaagggcgaggagctgttcaccggggtggtgatggcaccaagaagggctcgcggatgccggaccctcggtctcccggcgctgcttctgctgctgctgctccggccgccggcgacgcggggc

SEQ ID NO: 10 - DNA, 231 bp, Artificial Sequence
  CAR construct component
atcacgtgtcctccacccatgtccgtggaacacgcagacatctgggtcaagagctacagcttgtactccagggagcggtacatttgtaactctggtttcaagaggaaagccggcacgtccagcctgacggagtgcgtgttgaacaaggccacgaatgtcgcccactggacaacccccagtctcaaatgcattagagaccctgccctggttcaccaaaggccagcgccaccc

SEQ ID NO: 11 - DNA, 54 bp, Artificial Sequence
  CAR construct component
agcggcggaggatgtggaggcggaggctgtggcggaagcggaggcggaggcagt

SEQ ID NO: 12 - DNA, 354 bp, Artificial Sequence
  CAR construct component
atccagaactgggtgaatgtcataagtgatctgaagaaaattgaagatcttattcagtctatgcatattgatgctactctatatactgaaagtgatgttcaccccagttgcaaagtgacagcaatgaagtgctttctcttggagctgcaagttatttcacttgagtccggagatgcaagtattcatgatacagtggaaaatctgatcatccttgcaaacgacagtttgtcttctaatgggaatgtaacagaatctggatgcaaagaatgtgaggaactggaggaaaaaaatattaaagaatttttgcagagttttgtacatattgtccaaatgttcatcaacacttcttgataa

SEQ ID NO: 13 - DNA, 3749 bp, Artificial Sequence
  CAR construct
atgggatggagctgtatcatcctcttcctggtagcaacaacagccaccggcgtccatagcgagatcgtcctgactcagagccctgcaaccctgtccctgtctccaggcgaacgggctacactgagctgctctgcctctagcagcattaactacatttactggtatcagcagaagccaggccaagccccgaggctccttatttacttgacttccaacctggctagtggagtgccggctaggttcagcgggagcggctcaggcacagacttcacattgacaatatcaagcctggaacccgaagattttgctgtgtactattgtctccaatggtcttctaatcctctcacgttcggaggggggactaaggtggagattaaggggtcaacctctggcagcgggaaacctggctctggcgagggcagcaccaagggtcaagtccagctccaagaagtgcaactcgtggaatcaggaggcggcttggtgaagcccggcggctccttgagactgtcttgcgctgctagcgggtttacattctcatcttacgacatgagttgggttcgccaggcacctggaaaaggcctcgagtgggtcagcactatttctagcggcggatcttacacctactatctggacagcatcaaaggacgcttcacaatttctcgcgacaatgctaagaactctttgtatctccagatgaactcccttagagccgaggatactgctgtctactattgcgctcgccagggactcgattactggggacggggcacactcgtgactgtctcctctgtgcggccgccgagccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggaaatgaccaaaaaccaggtcagcctgacctgcctggtcaaaggccttctatccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaggatcccaaactctgctacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcatatgcaggctctgccacctagcggatccggcgcaacaaacttctctctgctgaaacaagccggagatgtcgaagagaatcctggaccgatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgttgtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggtgataatcccagtgtggtggtacggaattctctagactgccggatccaagctggaggaaaaactgtttcatacagaaggcgtggaggaaaaactgtttcatacagaaggcgtggaggaaaaactgtttcatacagaaggcgtggaggaaaaactgtttcatacagaaggcgtcgcgaattcgcggagactctagagggtatataatggaagctcgatttccagcttggcattccggtactgttggtaaacaccaagcttcaccatggtgagcaagggcgaggagctgttcaccggggtggtgatggcaccaagaagggctcgcggatgccggaccctcggtctcccggcgctgcttctgctgctgctgctccggccgccggcgacgcggggcatcacgtgtcctccacccatgtccgtggaacacgcagacatctgggtcaagagctacagcttgtactccagggagcggtacatttgtaactctggtttcaagaggaaagccggcacgtccagcctgacggagtgcgtgttgaacaaggccacgaatgtcgcccactggacaacccccagtctcaaatgcattagagaccctgccctggttcaccaaaggccagcgccacccagcggcggaggatgtggaggcggaggctgtggcggaagcggaggcggaggcagtatccagaactgggtgaatgtcataagtgatctgaagaaaattgaagatcttattcagtctatgcatattgatgctactctatatactgaaagtgatgttcaccccagttgcaaagtgacagcaatgaagtgctttctcttggagctgcaagttatttcacttgagtccggagatgcaagtattcatgatacagtggaaaatctgatcatccttgcaaacgacagtttgtcttctaatgggaatgtaacagaatctggatgcaaagaatgtgaggaactggaggaaaaaaatattaaagaatttttgcagagttttgtacatattgtccaaatgttcatcaacacttcttgataa

SEQ ID NO: 14 - protein, 20 aa, Artificial Sequence
  CAR construct component
MGWSCIILFLVATTATGVHS

SEQ ID NO: 15 - protein, 129 aa, Artificial Sequence
  CAR construct component
EIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKGSTSGSGKPGSGEGSTKGQVQLQ

SEQ ID NO: 16 - protein, 7 aa, Artificial Sequence
  CAR construct component
SSINYIY

SEQ ID NO: 17 - protein, 11 aa, Artificial Sequence
  CAR construct component
LLIYLTSNLAS

SEQ ID NO: 18 - protein, 8 aa, Artificial Sequence
  CAR construct component
LQWSSNPL

SEQ ID NO: 19 - protein, 119 aa, Artificial Sequence
  CAR construct component
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSVRPPS

SEQ ID NO: 20 - protein, 9 aa, Artificial Sequence
  CAR construct component
FTFSSYDMS

SEQ ID NO: 21 - protein, 15 aa, Artificial Sequence
  CAR construct component
WVSTISSGGSYTYYL

SEQ ID NO: 22 - protein, 6 aa, Artificial Sequence
  CAR construct component
RQGLDY

SEQ ID NO: 23 - protein, 231 aa, Artificial Sequence
  CAR construct component
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGLLSSDIAVEWESNGQPENNYKTTPPVLDSDGSFFLYSKLTVDKSRWQQGNVFSCSVMHEALHNHYTQKSLSLSPGK

SEQ ID NO: 24 - protein, 74 aa, Artificial Sequence
  CAR construct component
DPKLCYFWVLVVVGGVLACYSLLVTVAFIIFWVRSKRSRLLHSDYMNMTPRRPGPTRKHYQPYAPPRDFAAYRS

SEQ ID NO: 25 - protein, 23 aa, Artificial Sequence
  CAR construct component
WVLVVVGGVLACYSLLVTVAFII

SEQ ID NO: 26 - protein, 113 aa, Artificial Sequence
  CAR construct component
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPS

SEQ ID NO: 27 - protein, 22 aa, Artificial Sequence
  CAR construct component
GSGATNFSLLKQAGDVEENPGP

SEQ ID NO: 28 - protein, 198 aa, Artificial Sequence
  CAR construct component
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARG

SEQ ID NO: 29 - protein, 42 aa, Artificial Sequence
  CAR construct component
MVSKGEELFTGVVMAPRRARGCRTLGLPALLLLLLLRPPATR

SEQ ID NO: 30 - protein, 78 aa, Artificial Sequence
  CAR construct component
GITCPPPMSVEHADIWVKSYSLYSRERYICNSGFKRKAGTSSLTECVLNKATNVAHWTTPSLKCIRDPALVHQRPAPP

SEQ ID NO: 31 - protein, 18 aa, Artificial Sequence
  CAR construct component
SGGGCGGGGCGGSGGGGS

SEQ ID NO: 32 - protein, 116 aa, Artificial Sequence
  CAR construct component
IQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 33 - protein, 686 aa, Artificial Sequence
  CAR construct
MGWSCIILFLVATTATGVHSEIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKGSTSGSGKPGSGEGSTKGQVQLQEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSVRPPSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGLLSSDIAVEWESNGQPENNYKTTPPVLDSDGSFFLYSKLTVDKSRWQQGNVFSCSVMHEALHNHYTQKSLSLSPGKDPKLCYFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPS

SEQ ID NO: 34 - protein, 198 aa, Artificial Sequence; identical to SEQ ID NO: 28
  CAR construct component

SEQ ID NO: 35 - protein, 254 aa, Artificial Sequence
  CAR construct component
MVSKGEELFTGVVMAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSGGGCGGGGCGGSGGGGSIQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 36 - protein, 128 aa, Artificial Sequence
  CAR construct component
DIQMTQSPSSLSASVGDRVTITCSTSSSVSYMHWYQQKPGKAPRLLIYSTSNLASGVPSRFSGSGSGTDFTFTISSLQPEDIATYYCHQWSSYPTFGQGTKVEIKGSTSGSGKPGSGEGSTKGQVQLQ

SEQ ID NO: 37 - protein, 7 aa, Artificial Sequence
  CAR construct component
SSVSYMH

SEQ ID NO: 38 - protein, 11 aa, Artificial Sequence
  CAR construct component
LLIYSTSNLAS

SEQ ID NO: 39 - protein, 7 aa, Artificial Sequence
  CAR construct component
HQWSSYP

SEQ ID NO: 40 - protein, 120 aa, Artificial Sequence
  CAR construct component
ESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSTVTVSSGAAAE

SEQ ID NO: 41 - protein, 10 aa, Artificial Sequence
  CAR construct component
FTISSGYSWH

SEQ ID NO: 42 - protein, 13 aa, Artificial Sequence
  CAR construct component
WIGYIQYSGITNY

SEQ ID NO: 43 - protein, 12 aa, Artificial Sequence
  CAR construct component
REDYDYHWYFDV

SEQ ID NO: 44 - DNA, 744 bp, Artificial Sequence
  CAR construct component
gacatccagatgacccagagcccaagcagcctgagcgccagcgtgggtgacagagtgaccatcacctgtagtaccagctcgagtgtaagttacatgcactggtaccagcagaagccaggtaaggctccaaggctgctgatctacagcacatccaacctggcttctggtgtgccaagcagattcagcggtagcggtagcggtaccgacttcaccttcaccatcagcagcctccagccagaggacatcgccacctactactgccatcagtggagtagttatcccacgttcggccaagggaccaaggtggaaatcaaaggatccacttccggttcaggaaagcccgggagtggtgaaggtagcactaaaggccaggtccagctgcaggagagcggtccaggtcttgtgagacctagccagaccctgagcctgacctgcaccgtgtctggcttcaccatcagcagtggttatagctggcactgggtgagacagccacctggacgaggtcttgagtggattggatacatacagtacagtggtatcactaactacaacccctctctcaaaagtagagtgacaatgctggtagacaccagcaagaaccagttcagcctgagactcagcagcgtgacagccgccgacaccgcggtctattattgtgcaagagaagactatgattaccactggtacttcgatgtctggggtcaaggcagcacggtcaccgtctcctcaggtgcggccgccgag